(19) United States
(12) Patent Application Publication
(10) Pub. No.: US 2023/0087609 A1
(43) Pub. Date: Mar. 23, 2023

(54) USE OF A SOLUBLE GUANYLATE CYCLASE (sGC) STIMULATOR OR OF A COMBINATION OF A sGC STIMULATOR AND AN sGC ACTIVATOR FOR CONDITIONS WHEREIN THE HEME GROUP OF sGC IS OXIDIZED OR WHEREIN sGC IS DEFICIENT IN HEME

(71) Applicants: Universiteit Maastricht, Maastricht (NL); Academisch Ziekenhuis Maastricht, Maastricht (NL)

(72) Inventors: Harald Horst Heinz Wilhelm Schmidt, Aachen (DE); Mahmoud Hassan Mahmoud Elbatrik, Maastricht (NL); Thao-Vi Dao, Frankfurt (DE); Ana Isabel Casas Guijarro, Maastricht (NL); Theodora Saridaki, Aachen (DE); Alexandra Petraina, Maastricht (NL)

(73) Assignees: Universiteit Maastricht, Maastricht (NL); Academisch Ziekenhuis Maastricht, Maastricht (NL)

(21) Appl. No.: 17/797,448

(22) PCT Filed: Feb. 18, 2021
(86) PCT No.: PCT/NL2021/050111

§ 371 Date: Aug. 4, 2022

(30) Foreign Application Priority Data
Feb. 21, 2020 (NL) 2024965

(51) Int. Cl. A61K 31/506; A61K 31/137; A61K 31/197; A61K 31/416; A61K 31/427; A61K 31/4439; A61K 31/5377; A61K 45/06; A61P 9/00

(52) U.S. Cl. CPC A61K 31/506; A61K 31/137; A61K 31/197; A61K 31/416; A61K 31/427; A61K 31/4439; A61K 31/5377; A61K 45/06; A61P 9/00

(57) ABSTRACT

The invention relates to a pharmaceutical composition comprising one or more stimulators of soluble guanylate cyclase (sGC), or a combination of at least one stimulator of sGC and at least one activator of sGC, for use in a method of treatment of a disease and/or a disorder that is/are associated with a deficiency of cyclic 3′,5′-guanosine monophosphate in the patient to be treated. The invention also relates to a therapeutic combination comprising a first unit dose comprising an sGC stimulator and a second unit dose comprising an sGC activator. The invention also relates to a therapeutic combination comprising a first unit dose comprising a first sGC stimulator and a second unit dose comprising a second sGC stimulator, for use in a method for the treatment of a disease and/or a disorder that is/are associated with a deficiency of cyclic 3′,5′-guanosine monophosphate in the patient to be treated. Furthermore, the invention relates to a kit comprising a pharmaceutical composition comprising one or more stimulators of sGC; a pharmaceutical composition comprising one or more stimulators of sGC and one or more activators of sGC; a therapeutic combination comprising a first unit dose comprising a first sGC stimulator and a second unit dose comprising a second sGC stimulator; or to a therapeutic combination comprising a first unit dose comprising an sGC stimulator and a second unit dose comprising an sGC activator.

### TECHNOLOGICAL FIELD

The present invention relates to a method for treating a disease and/or a disorder that is/are associated with a deficiency of cyclic 3′,5′-guanosine monophosphate (cGMP), by administering an effective dose of a pharmaceutical composition comprising one or more stimulators of soluble guanylate cyclase (sGC) to a patient in need thereof, or by administering an effective dose of a combination of at least one stimulator of sGC and at least one activator of sGC to a patient in need thereof. The invention also relates to a pharmaceutical composition comprising one or more stimulators of sGC, or a combination of at least one stimulator of sGC and at least one activator of sGC, for use in a method of treatment of a disease and/or a disorder that is/are associated with a deficiency of cyclic 3′,5′-guanosine monophosphate in the patient to be treated. The invention also relates to a therapeutic combination comprising a first unit dose comprising an sGC stimulator and a second unit dose comprising an sGC activator. The invention also relates to a therapeutic combination comprising a first unit dose comprising a first sGC stimulator and a second unit dose comprising a second sGC stimulator, for use in a method for the treatment of a disease and/or a disorder that is/are associated with a deficiency of cyclic 3′,5′-guanosine monophosphate in the patient to be treated. Furthermore, the invention relates to a kit comprising a pharmaceutical composition comprising one or more stimulators of sGC; a pharmaceutical composition comprising one or more stimulators of sGC and one or more activators of sGC; a therapeutic combination comprising a first unit dose comprising a first sGC stimulator and a second unit dose comprising a second sGC stimulator; or to a therapeutic combination comprising a first unit dose comprising an sGC stimulator and a second unit dose comprising an sGC activator. An aspect of the invention relates to a therapeutic combination comprising: a first unit dose comprising: a first sGCs; optionally a second sGCs; and either a second unit dose comprising: a first sGCa; optionally a second sGCa; or a third unit dose comprising: a third sGCs; and optionally a fourth sGCs.

### BACKGROUND ART

Cyclic 3′,5′-guanosine monophosphate (cGMP), one of the most important mediators in eukaryotic organisms, is formed by guanylate cyclases (GC) that catalyze the conversion of guanosine triphosphate (GTP) to 3′,5′-cyclic guanosine monophosphate (cGMP) and pyrophosphate. One important isoform is soluble guanylate cyclase (sGC), an intracellular heterodimeric enzyme that is regulated by endogenous nitric oxide (NO) or NO donor compounds. By binding to sGC's histidine-ligated Fe(II)-heme, NO induces cleavage of the proximal histidine-Fe(II) bond and a subsequent conformational change in sGC that activates the enzyme's catalytic site, increasing its GTP to cGMP conversion rate by up to two orders of magnitude. Other domains of the sGC protein can be pharmacologically targeted by so-called sGC stimulator compounds (abbreviated to “sGCs” for an sGC stimulator, an sGC stimulator compound, which both have the same meaning here and throughout the text) that allosterically enhance sGC's apparent affinity for NO or its efficacy to stimulate cGMP formation. Oxidation or loss of sGC's heme, e.g. under disease conditions, yields so-called apo-sGC (Horst & Maletta, Nitric Oxide. 2018; 77:65-74. doi: 10.1016/j.niox.2018.04.011; Montfort et al., Antioxid Redox Signal. 2017; 26(3):107-121. doi: 10.1089/ars.2016.6693), which is no longer responsive to NO and represents a disruption of NO-cGMP signalling. NO-sGC-cGMP signalling is implicated in a large number of biological pathways that involve protein kinases and ion channels. cGMP deficiency is therefore of high pathological significance in many diseases.

It follows from the above that insufficient cGMP synthesis is attributed to two different conditions. First, NO levels may be suboptimal for various reasons (insufficient NO synthesis or enhanced breakdown of NO by reactive oxygen species). In this case, sGCs are used clinically and thought to allosterically regulate sGC to increase basal activity and to make it more sensitive for NO so that, despite lower than normal levels of NO, normal cGMP levels are formed. The current understanding is that their effect depends on sGC having an intact and properly ligand-bound heme. One prominent example of such sGC stimulators is riociguat, used to treat pulmonary arterial hypertension (Mittendorf et al., ChemMedChem. 2009; 4(5):853-865. doi: 10.1002/cmdc.200900014). Other compounds with this activity are under investigation for heart failure, with vericiguat being approved by the FDA for use in the treatment of heart failure. See also Table 3, here below, summarizing recent phase II and phase III clinical trials.

Second, sGC may lose its heme through oxidative damage (as it is thought to occur under oxidative stress and in reperfusion injury following ischemia) or other chemical reactions, resulting in conversion of a significant fraction of the enzyme to so-called apo-sGC, which is considered insensitive to NO. Here, so-called sGC activator compounds (referred to as “sGCa” or “apo-sGCa” for an sGC activator, an sGC activator compound, which both have the same meaning throughout the text) are used therapeutically. These are believed to bind to the empty heme pocket of apo-sGC, mimic the heme in its NO-bound state (i.e. without the proximal histidine ligated to the heme iron) and thereby restore the protein conformation, which is attained when NO binds heme-containing sGC, thereby allowing cGMP formation to fully resume as in physiological NO-activated sGC. Such heme-independent compounds are under investigation for acute and chronic conditions where high levels of reactive oxygen species play a central role, such as ischemic stroke, myocardial infarction, heart failure, and sickle cell disease. Cinaciguat is a prominent representative of this class (Kollau et al., Mol Pharmacol. 2018; 93(2):73-78. doi: 10.1124/mol.117.109918). See also Table 4, here below, summarizing recent phase II clinical trials.

Both compound classes, i.e. sGC stimulators (sGCs) and sGC activators (sGCa), are thought to act in a mutually exclusive manner on sGC and apo-sGC, respectively, according to current principles and dogma generally accepted nowadays by the skilled persons of the relevant technological field (“Soluble Guanylate Cyclase Stimulators and Activators”, Sandner, P., Zimmer, D. P., Todd Milne, G., Follmann, M., Hobbs, A., Stasch, J-.P., Handbook of Experimental Pharmacology, 2018, pp.1-40, Springer Nature Switzerland AG, doi number is 10.1007/164_2018_197, first online at 29 Jan. 2019). See also Table 1 for an overview of compounds assigned to either the group of compounds acting as sGCs, or the group of compounds acting as sGCa. Therefore, each of the two compound classes is investigated mostly for a relatively narrow range of medical conditions (NO deficiency or oxidative damage, respectively), and treatment of each of the two medical conditions requires relatively high doses of the respective compounds.

There is a demand for sGC modulator treatment regimens for use in methods of treating a broad range of medical conditions related to sGC dysfunction and cGMP deficiency.

TABLE 1
Current taxonomy of sGC modulating drugs

| | sGC | Apo-sGC |
|---|---|---|
| Compound | stimulators | activators (sGCa, |
| class | (sGCs) | or apo-sGCa) |
| Examples | Riociguat, Vericiguat | Cinaciguat |
| | BAY 60-4552 | HMR 1766 |
| | YC-1 (lificiguat) | BI 703704 |
| | A-350619 | BI 684067 |
| | CF-1571 | |
| Target protein | heme-containing sGC | Apo-sGC |
| Binding site | Allosteric binding site | Heme binding site |
| Mechanism | Direct sGC stimulation | Direct activation |
| | and sensitization | of apo-sGC |
| | of sGC for NO | |
| Indication | Conditions with | Conditions where |
| | reduced NO | sGC is oxidatively |
| | levels | damaged or for other |
| | | reasons heme-free |

### SUMMARY

The present invention relates to the surprising finding that the current concept of dichotomous mechanisms of action of sGCs/sGC and sGCa/apo-sGC is wrong. Instead, sGC stimulators are equally effective on apo-sGC and sGC, meaning that these sGCs are suitable for therapeutic use either alone or for application in therapeutic regimen in a synergistic manner together with sGC activators, optionally at a lower dose of the sGCs and/or the sGCa than currently applied or investigated, and/or optionally at lower concentrations of both the sGCs and the sGCa. This surprising finding results in, firstly, a better mechanistic understanding of both compound classes sGCs and sGCa, that is highly relevant and important for selection of disease- and mechanism-based patient stratification, secondly, use extension of sGC stimulators, thirdly, new drug combinations, and fourthly, a lower risk of dose/concentration-dependent side effects, to name a few of the many advantages now achieved with the present invention. Thus, the revised taxonomy of sGC modulation drugs is as outlined in Table 2. Combinations of one or more sGCs compounds or combinations of one or more sGCs compounds combined with one or more sGCa compounds are thus suitable for use in a method for the treatment of a disease or disorder associated with a deficiency of 3′,5′-guanosine monophosphate, such as any of the cardiovascular diseases tabulated in Tables 1-4.

An aspect of the invention relates to a therapeutic combination comprising:
a. a first unit dose comprising:
   i. a first sGCs;
   ii. optionally a second sGCs; and either
b. a second unit dose comprising:
   i. a first sGCa;
   ii. optionally a second sGCa; or
c. a third unit dose comprising:
   i. a third sGCs; and
   ii. optionally a fourth sGCs.

TABLE 2
Revised taxonomy of sGC modulating drugs, based on the insights
and details provided by the current invention

| | | Apo-sGC |
|---|---|---|
| Compound | sGC stimulators | activators (sGCa, |
| class | (sGCs, or apo-sGCs)^{‡} | or apo-sGCa) |
| Examples | Riociguat | Cinaciguat |
| | Vericiguat | HMR 1766 |
| | BAY 60-4552 | BI 703704 |
| | YC-1 (lificiguat) | BI 684067 |
| | A-350619 | |
| | CF-1571 | |
| Target | heme-containing | Apo-sGC |
| protein | sGC and Apo-sGC | |
| Binding site | Allosteric binding site | Heme binding site |
| Mechanism | Direct sGC stimulation | Direct activation |
| | and sensitization of sGC | of apo-sGC |
| | for NO as well as direct | |
| | stimulation of apo-sGC | |
| | as well as sensitization | |
| | of apo-sGC for sGCa | |
| Indication | Conditions with reduced | Conditions |
| | NO levels and Conditions | where sGC is |
| | where sGC is oxidatively | oxidatively damaged |
| | damaged or for other | or for other |
| | reasons heme-free | reasons heme-free |

^{‡}Based on the surprising findings by the inventors, that compounds currently generally known as sGC stimulators are also endowed with the activity to activate/stimulate apo-sGC, the inventors also refer to the currently known sGCs compounds as “apo-sGCs”. For the sake of clarity, in the description and in the claims, the currently common nomenclature is maintained, however: sGCs and sGCa (See for example: “Soluble Guanylate Cyclase Stimulators and Activators”, Sandner, P., Zimmer, D.P., Todd Milne, G., Follmann, M., Hobbs, A., Stasch, J-.P., Handbook of Experimental Pharmacology, 2018, pp.1-40, Springer Nature Switzerland AG, doi number is 10.1007/164_2018_197, first online at 29 Jan. 2019).

NB: Both compound classes can be combined in a synergistic manner, allowing lower doses of both, according to the invention and as demonstrated in exemplifying embodiments of the invention (see the Examples section, here below).

Scheme 1 and Scheme 2 (here below) illustrate the difference between the dogma currently accepted in the field and the new concepts, now having become apparent based on the new insights provided by the current invention.

Scheme 1: Before the present invention, sGC stimulators (sGCs) and sGC activators (sGCa) were thought to have distinct targets; sGC stimulators stimulate heme containing sGC and augment its stimulation by NO; sGC activators activate apo-sGC, which is heme-free and insensitive to NO. sGC stimulators were considered inactive/irrelevant with respect to apo-sGC.

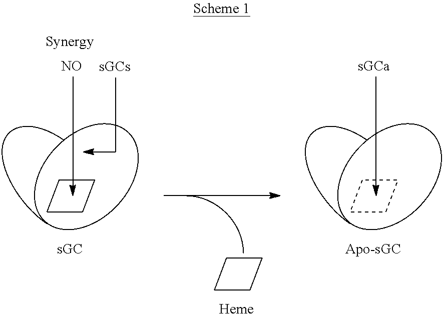

Scheme 2 describes the concept on the use of sGC stimulators and sGC activators after the present invention. sGC stimulators act on both heme containing sGC (which they directly stimulate and augment its stimulation by NO) and on apo-sGC. In both cases the sGCs compounds directly stimulate and augment the stimulation/activation by NO and sGC activators, respectively. Thus, the use of sGC stimulators can be extended to conditions where apo-sGC is present or even increased as a mechanism of disease, and to combinations with sGC activators.

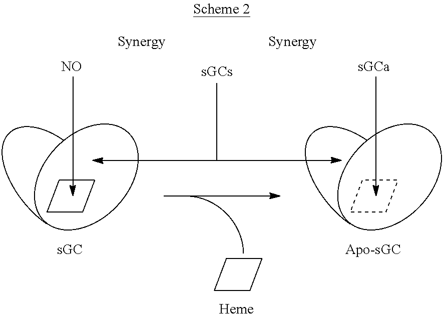

An aspect of the invention relates to a pharmaceutical composition comprising a soluble guanylate cyclase (sGC) stimulator compound (sGCs) and an sGC activator compound (sGCa).

A further aspect of the invention relates to an oral dose combination comprising a first oral dose comprising an sGCs and a second oral dose comprising an sGCs, the first oral dose and the second oral dose optionally comprising one or more pharmaceutically acceptable excipient(s).

An aspect of the invention relates to a kit comprising the pharmaceutical composition of the invention or the oral dose combination of the invention, and optionally instructions for use.

An aspect of the invention relates to a pharmaceutical composition of the invention or oral dose combination of the invention, for use as a medicament. An aspect of the invention relates to pharmaceutical composition of the invention or oral dose combination of the invention for use in a method for the treatment of cyclic 3′,5′-guanosine monophosphate (cGMP) deficiency in a patient, preferably a human patient.

An aspect of the invention relates to a pharmaceutical composition or oral dose combination for use according to the invention, wherein the use is in a method for the treatment of a cardiovascular disease, or wherein the patient deficient in cGMP suffers from a cardiovascular disease.

An aspect of the invention relates to a pharmaceutical composition comprising at least two sGCs for use as a medicament.

An aspect of the invention relates to an oral dose combination comprising a first oral dose comprising a first sGCs and a second oral dose comprising a second sGCs, the first oral dose and the second oral dose optionally comprising one or more pharmaceutically acceptable excipient(s), for use according to the invention.

An embodiment is the pharmaceutical composition or oral dose combination for use according to the invention, wherein the use is in a method for the treatment of cyclic 3′,5′-guanosine monophosphate (cGMP) deficiency in a patient, preferably a human patient.

An aspect of the invention relates to a therapeutic combination comprising:
a. a first unit dose comprising:
   i. a first sGCs;
   ii. optionally a second sGCs; and
b. a second unit dose comprising:
   i. a first NO donor compound;
   ii. optionally a second NO donor compound; and optionally comprising
c. a third unit dose comprising:
   i. a first sGCa; and
   ii. optionally a second sGCa.

An aspect of the invention relates to the therapeutic combination of the invention for use as a medicament, wherein the therapeutic combination comprises:
a. a first unit dose comprising:
   i. a first sGCs;
   ii. optionally a second sGCs; and
b. a second unit dose comprising:
   i. a first NO donor compound;
   ii. optionally a second NO donor compound; and optionally comprises
c. a third unit dose comprising:
   i. a first sGCa; and
   ii. optionally a second sGCa.

### BRIEF DESCRIPTION OF DRAWINGS

The present invention will be discussed in more detail below, with reference to the attached drawings, in which the following is displayed:

FIG. 1: sGC activity in lung homogenates of apo-SGC mice either stimulated with the sGC activator BAY 58-2667 at 0.3 μM (open bar); with sGC stimulator BAY 41-2272 at 10 μM (black bar), or with a combination of both compounds at the respective concentrations (hatched bar). The specific activity of sGC is expressed as fold stimulation versus basal activity. *P<0.05 one-way analysis of variance (ANOVA); data represents means±standard error mean from n=3, with two replicates each.

FIG. 2: Maximal relaxation of isolated rat mesenteric arteries in response to the sGC activator BAY 58-2667 alone (0.0003 μM, open bar) and in the presence of the sGC stimulator BAY 41-2272 at 0.1 μM (black bar). BAY 41-2272 alone showed no relaxation. Values are expressed as a percentage reversal of the level of pre-contraction to U46619, with the response to 10 μM nifedipine defined as 100% relaxation. *P<0.05 Student's t-test; data represents means ±standard error mean from n=2, with two replicates.

FIG. 3: The same experimental setup as in FIG. 2 was employed but with BAY 58-2667 at 0.001 μM alone (open bar) and in the presence of 0.1 μM BAY 41-2272 (black bar).

FIG. 4: The same experimental setup as in FIGS. 2 and 3 was employed but with BAY 58-2667 at 0.003 μM alone (open bar) and in the presence of 0.1 μM BAY 41-2272 (black bar).

FIG. 5: sGC activity in lung homogenates of apo-SGC mice either stimulated with the sGC activator BAY 60-2770 at 0.1 μM (open bar); with sGC stimulator BAY 41-2272 at 1 μM (black bar), or with a combination of both compounds at the respective concentrations (hatched bar). The specific activity of sGC is expressed in nmol/mg/min versus basal activity. *P<0.05 one-way analysis of variance (ANOVA); data represents means±standard error mean from three replicates of one animal.

FIG. 6: The same experimental setup as in FIG. 5 was employed but with BAY 60-2770 at 0.3 μM alone (open bar);

FIG. 7A: sGC activity of purified human sGC (EnzoLifeScience, Lörrach, Germany) treated with 10 μM ODQ (ODQ oxidates the heme group of the sGC), and stimulated with the sGC activator BAY 58-2667 at 0.3 μM alone (open bar); and in presence of sGC stimulator BAY 41-2272 at 10 μM (black bar). The specific activity of sGC is expressed in μmol/mg/min. *P<0.05 Student's-t-test; data represents means±standard error mean from three experiments.

FIG. 7B: dose response curve for the purified human sGC treated with a concentration series of ODQ in the presence of 30 μM of the sGC stimulator Bay 41-2272.

FIG. 8: sGC activity of human apo-sGC expressed in SF-9 cells stimulated with the sGC stimulator BAY 41-2272 at 10 μM alone (open bar); and in presence of ODQ at 10 μM (black bar). The specific activity of sGC is expressed in nmol/mg/min. *P<0.05 Student's-t-test; data represents means±standard error mean from three experiments.

FIG. 9: sGCs in the apo-sGC in vitro disease model, stroke. Human Brain Microvascular Endothelial Cells (HBMECs) were subjected to hypoxia and treated with BAY 41-2272 (‘BAY41’, 1 μM). sGCs treatment (grey bar, right) significantly increased cell viability and restored cell viability to a level comparable to control cells that were untreated (left bar, white) and in comparison with non-treated cells, which were treated with OGD (black bar, middle). ^{###}P<0.001 and **p<0.01 Student's-t-test; data represents means ±standard error mean from six experiments.

FIG. 10: Post-stroke treatment with BAY 63-2521 (riociguat, approved as Adempas) reduces infarct size in a stroke animal model. Adult mice were subjected to 1 h transient occlusion of the middle cerebral artery (tMCAO) followed by 24 h of reperfusion. 1 h post-reperfusion treatment with the sGCs reduced infarct volume (grey bar, right) in comparison to non-treated animals (black bar, left), and *P<0.05 Student's-t-test; data represents means ±standard error mean from six experiments.

FIG. 11: sGC activity in lung homogenates of apo-sGC mice stimulated with the sGC stimulator BAY 41-2272 at 10 μM in presence (open bar, left); and in absence of ODQ at 10 μM (black bar, right).

FIG. 12: A. The nitric oxide donor DETA-NONOate at 10 micromole/L concentration was added to control cells 1 (left bar, white) and to test cells (right, black bar); to the control cells 2 (middle bar, gray) and to the test cells, 30 micromole/L of Bay41-2272 was added, resulting in an increase in cGMP formation for the control cells 2; thus, to the test cells (right bar, black), combination of 30 micromole/L of Bay41-2272 and 10 micromole/L of the NO donor compound were added, resulting in a synergistic increase in cGMP formation, compared to control cells 1 (NO donor compound only) and compared to control cells 2 (Bay41-2272, only). B. The nitric oxide donor DETA-NONOate at 10 micromole/L concentration was added to control cells (left bar) and to test cells (right, black bar); to the test cells, also 30 micromole/L of Bay41-2272 was added, resulting in an increase in cGMP formation. C. The nitric oxide donor DETA-NONOate at 100 micromole/L concentration and Bay 41-2272 at 30 micromole/L concentration were added to control cells (left bar) and to test cells (right, black bar); to the test cells, also 0,1 micromole/L of Bay 60-2770 was added, resulting in a similar extent of cGMP formation compared to the control cells (left bar, gray).

### DETAILED DESCRIPTION

It is a first goal of the present invention to provide improved therapy options for patients suffering from any health threatening or even life-threatening condition accompanied with the presence of apo-sGC in the patient's tissue cells.

It is an objective of the current invention to provide pharmaceutical compositions suitable for the treatment of diseases and impaired health conditions relating to dysfunctional sGC and/or apo-sGC, e.g. cardiovascular diseases and disorders, sickle cell disease, etc.

At least one of the above objectives is achieved by providing at least one sGCs alone or in combination with at least one sGCa for the treatment of cardiovascular disease accompanied with apo-sGC in the patients suffering from said cardiovascular disease (CVD).

At least a further objective is achieved by providing a method for treating cardiovascular disease patients wherein the treatment encompasses the step of administering an effective dose of at least one sGCs to a CVD patient in need thereof, wherein the patient has a CVD related to the presence of apo-sGC, and wherein optionally the step of administering an effective dose of at least one sGCa to said CVD patient in need thereof. Preferably and beneficially, the (daily) dose of the sGCs compound(s) and/or the sGCa compound(s) is/are lower than the dose(s) that would be administered to said CVD patient when only the sGCa would be administered or when the one or more sGCs would be administered to a CVD patient suffering from a disease or impaired health relating to presence of reduced NO levels in the patient.

The inventors surprisingly have found that, contrary to current belief by the skilled person in the relevant technological field, sGC that has lost its heme group still retains a functional allosteric binding site for sGC stimulator compounds and still remains sensitive to such stimulators. The current inventors now established that current widely accepted teaching—i.e., the notion that oxidizing the sGC heme moiety or removing the sGC heme moiety results in a mandatory loss of responsiveness to sGC stimulator compounds—has resulted from producing apo-sGC using agents that also render the sGC stimulator site unresponsive to stimulation. The current inventors now established and demonstrated that sGC stimulator-responsive apo-sGC is produced by converting sGC to apo-sGC with agents that cause the heme moiety of sGC to dissociate from the protein but without damaging the sGC stimulator binding site. The apo-sGC thus produced resembles the naturally occurring apo-sGC of patients suffering from a disease or aberrancy related to cyclic 3′,5′-guanosine monophosphate deficiency.

Without wishing to be bound by theory, the inventors established that the current belief that formation of apo-sGC would invariably lead to the loss of responsiveness to sGC stimulator compounds resulted from the general use in the field of drug discovery, of 1H-[1,2,4]oxadiazolo[4,3-a]quinoxalin-1-one (ODQ), which compound is considered to be a highly selective, irreversible, heme-site inhibitor of soluble guanylyl cyclase competitive with NO, to generate apo-sGC. However, in contrast to what is generally believed and accepted, the current invention now surprisingly makes clear that ODQ also damages the sGC stimulator binding site of apo-sGC, thereby artificially generating forms of the enzyme that do not predominate in vivo, i.e. both apo- and oxidized at the allosteric site. Importantly, if one generates apo-sGC without such use of chemical artefact as above, e.g. by mutating the heme-binding amino-acid histidine without using ODQ, this form of pure and truly apo-sGC is apparently fully sensitive to sGC stimulators according to the invention and shows pharmacological synergy between representatives of the two compound classes sGCs and sGCa according to the invention.

### Definitions

As used herein, the terms “treatment,” “treating” and the like, refer to obtaining a desired pharmacologic and/or physiologic effect by administering a therapeutically effective dose of one or more pharmacologically active compounds in the form of a pharmaceutical composition. “Treatment,” as used herein, covers any treatment of an undesirable medical condition in a subject, particularly in a human, and includes: (a) preventing the condition from occurring in a subject which may be predisposed to the disease but has not yet been diagnosed as having it, or in a subject that is expected to be exposed to events that are likely to precipitate the condition; (b) inhibiting the progression of medical condition, i.e., slowing or arresting its development; and (c) relieving the condition, e.g., causing regression of the condition, e.g., to completely or partially remove symptoms of the condition.

The term “therapeutically acceptable amount” or “therapeutically effective dose” interchangeably refer to an amount sufficient to affect the desired result. In some embodiments, a therapeutically acceptable amount does not induce or cause undesirable side effects. A therapeutically acceptable amount can be determined by first administering a low dose, and then incrementally increasing that dose until the desired effect is achieved.

As used herein, the terms “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus <10% of the particular term and “substantially” and “significantly” will mean plus or minus >10% of the particular term.

The present invention will be described with respect to particular embodiments but the invention is not limited thereto but only by the claims.

Furthermore, the terms first, second, third and the like in the description and in the claims, are used for distinguishing between similar elements and not necessarily for describing a sequential or chronological order. The terms are interchangeable under appropriate circumstances and the embodiments of the invention can operate in other sequences than described or illustrated herein.

The embodiments of the invention described herein can operate in combination and cooperation, unless specified otherwise.

Furthermore, the various embodiments, although referred to as “preferred” or “e.g.” or “for example” or “in particular” are to be construed as exemplary manners in which the invention may be implemented rather than as limiting the scope of the invention.

The term “comprising”, used in the claims, should not be interpreted as being restricted to the elements or steps listed thereafter; it does not exclude other elements or steps. It needs to be interpreted as specifying the presence of the stated features, integers, steps or components as referred to, but does not preclude the presence or addition of one or more other features, integers, steps or components, or groups thereof. Thus, the scope of the expression “a composition comprising compounds A and B” should not be limited to a composition consisting only of compounds A and B, rather with respect to the present invention, the only enumerated compounds of the composition are compound A and compound B, and further the claim should be interpreted as including equivalents of those compounds.

In addition, reference to an element by the indefinite article “a” or “an” does not exclude the possibility that more than one of the element are present, unless the context clearly requires that there is one and only one of the elements. The indefinite article “a” or “an” thus usually means “at least one”.

While the invention has been described in terms of several embodiments, it is contemplated that alternatives, modifications, permutations and equivalents thereof will become apparent to one having ordinary skill in the art upon reading the specification. The invention is not limited in any way to the illustrated embodiments and figures. Changes can be made without departing from the scope which is defined by the appended claims.

### Compositions of the Invention

Compositions of the invention include at least one sGC stimulator and/or at least one sGC stimulator combined with at least one sGC activator, wherein the sGCs and the sGCa is/are (all) in the form of pharmaceutically acceptable salts such as salts that are generally known in the art, and in the case of the present invention, include (relatively) non-toxic, organic or inorganic salts of the compounds of the present invention. Examples of such salts include, but are not limited to, acid addition salts; basic salts such as alkali metal salts, alkaline earth salts, and ammonium salts; or organic salts may also be used included, e.g., salts of lysine, choline, diethanolamine, ethylenediamine, meglumine (N-methylglucamine), procaine and organic pH buffer compounds.

Furthermore, compositions of the invention are compositions comprising co-crystals of an sGCs and an sGCa wherein the sGCs and the sGCa are co-crystallized such that in the crystals the sGCs and sGCa are present in a ratio of 1(:)1. This way, administering a pharmaceutical composition of the invention comprising such a co-crystal of an sGCs and an sGCa in a 1(:)1 molar ratio, results in the provision of a dose of sGCs and a dose of sGCa to the patient, wherein the two doses are essentially equal.

The sGCs and sGCa compounds as used in this invention or for use in a method for the treatment of a disease such as a CVD relating to sub-optimal NO concentration in the patient and/or relating to the presence of apo-sGC, of the invention, or salts of said compounds, optionally exist in the form of solvates. As used herein, the term “solvate” refers to a complex of variable stoichiometry formed by a solute or a salt thereof, and a solvent. Such solvents for the purpose of the invention do not interfere with the biological activity of the solute. Examples of suitable solvents include water, methanol, ethanol and acetic acid. If the solvent used is water, the solvate is for example referred to as a hydrate.

Compositions of the invention optionally contain stabilizers, preservatives, wetting- and emulsifying agents, consistency-improving agents, flavor-improving agents, solubilizers, colorants and masking agents and antioxidants as pharmaceutical adjuvants.

In some embodiments, one or more compounds selected from the class of sGC stimulators and/or one or more compounds selected from the class of sGCa activators are provided as a prodrug that is inactive or minimally active towards sGC and/or towards apo-sGC, and such compound(s) is/are after administration metabolized or otherwise converted to a biologically active or more active compound with respect to its sGC-related property. Optionally, such a prodrug has, relative to the active drug, altered metabolic stability or transport characteristics, fewer side effects or lower toxicity, or improved flavor.

Pharmaceutical compositions adapted for oral administration are for example presented as discrete units such as capsules or tablets; as powders or granules; as solutions, syrups or suspensions (in aqueous or non-aqueous liquids; or as edible foams or whips; or as emulsions). Such pharmaceutical compositions are for example solid, semi-solid, or liquid and typically comprise, in addition to the active ingredient or ingredients (active pharmaceutical ingredient(s), e.g. one or more sGCs, one or more sGCa), at least one pharmaceutically acceptable solvent or excipient; for example, a pharmaceutically acceptable carrier and/or a pharmaceutically acceptable diluent. Suitable carriers and/or diluents are well known in the art and include pharmaceutical grade starch, mannitol, lactose, magnesium stearate, sodium saccharin, talcum, cellulose, glucose, sucrose (or other sugar), magnesium carbonate, gelatin oil, alcohol, detergents, emulsifiers or water (preferably sterile). Oral compositions contain for example the active ingredients of the invention at doses of 0.1 mg to 1000 mg, preferably at doses of 1-500 mg, most preferably at doses of 5-250 mg. For example, a pharmaceutical composition of the invention (for use in a method for the treatment of a disease or disorder associated with a deficiency of 3′,5′-guanosine monophosphate) comprises 0.1 mg-5 mg of the sGCs riociguat (BAY 63-2521) and/or 0.05 mg-2 mg of the sGCs nelociguat (BAY 60-4552) and/or 0.1 mg-30 mg of the sGCs vericiguat (BAY 102-1189) and/or 0.1 mg-10 mg of the sGCs olinciguat (IW-1701) and/or 1 mg-100 mg of the sGCs praliciguat (IW-1973), or any combination thereof, for example combined with an sGCs such as for example 5 mg-400 mg of sGCa ataciguat (HMR 1766).

For example, an oral dose combination of the invention comprises a first oral dose comprising 0.1 mg-5 mg of the sGCs riociguat (BAY 63-2521) and/or 0.05 mg-2 mg of the sGCs nelociguat (BAY 60-4552) and/or 0.1 mg-30 mg of the sGCs vericiguat (BAY 102-1189) and/or 0.1 mg-10 mg of the sGCs olinciguat (IW-1701) and/or 1 mg-100 mg of the sGCs praliciguat

(IW-1973), or any combination thereof, and a second oral dose comprising e.g. an sGCs such as for example 5 mg-400 mg of sGCa ataciguat (HMR 1766).

Pharmaceutical compositions adapted for parenteral administration will be administered by injection (subcutaneously, intramuscularly or intravenously), or by intravascular infusion. Such compositions will include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Parenteral compositions will contain the active ingredients of the invention at doses of 0.01 mg/ml to 10 mg/ml.

Pharmaceutical compositions adapted for transdermal administration may be formulated as ointments, creams, suspensions, lotions, powders, solutions, pastes, gels, sprays, or oils; or they may be integrated into transdermal therapeutic systems (commonly known as transdermal patches), which will optionally contain dermal or mucosal penetration enhancers. Such transdermal systems will typically deliver 10 -100 mg of the compounds of the invention over a period of 24-72 hours.

Pharmaceutical compositions adapted for nasal administration may be provided as sprayable liquid or as finely dispersed solid. Pernasal compositions that are solid will be powders having a particle size for example in the range 20-500 μM which is administered into the nasal passage from a container of the powder held close up to the nostrils, or has an outlet that will be inserted into the nostrils for inhalation. Suitable compositions wherein the carrier is a liquid, for administration as a nasal spray or as nasal drops, include aqueous or oil solutions of the active ingredient.

Pharmaceutical compositions adapted for topical ocular administration may be formulated as eye drops or ocular gels as are known in the art of ocular drug delivery. Such compositions will contain the compounds of the invention at concentrations of about 0.001-3% (w/v), preferably about 0.01-1% (w/v), added to a basal medium to make an aqueous solution or a gel. The pH of the eye-drops of this invention is adjusted to about 4 to 10, preferably about 5 to 9.

The compositions of the invention optionally further comprise one or more additional therapeutic agents that are known to be effective in the context of the therapeutic indication.

A pharmaceutical composition of the invention is for example provided in unit dosage form, and is generally provided in a sealed container and is for example provided as part of a kit. Such a kit optionally (although not necessarily) includes instructions for use. A kit according to the invention includes a single unit dosage form or includes a plurality of said unit dosage forms.

### Dosing

The appropriate amount and frequency of administration of the compounds of the invention is determined according to the judgment of the attending clinician considering such factors as the type and severity of the disease and the characteristics of the individual, such as general health, age, sex, body weight and tolerance to drugs. The skilled artisan is able to determine appropriate dosages depending on these and other factors.

### Embodiments

In one embodiment, the pharmaceutical composition contains one or more sGC stimulators. In a preferred embodiment, the pharmaceutical composition contains one or more sGC stimulators and one or more sGC activators.

In one exemplary, non-limiting embodiment, the sGC stimulator is selected from any one or more of riociguat (BAY63-2521), vericiguat (BAY1021189/MK-1242-001), nelociguat (desmethyl riociguat), olinciguat (IW-1701), BAY41-2272, BAY60-4552, IWP-953, A-350619, CF-1571, CFM-1571, lificiguat (YC-1), etriciguat, praliciguat (IW-1973), any one or more of the compounds disclosed in published international patent applications WO/2000/06568, WO/2000/06569, WO/2002/42301, WO/2003/095451, WO/2011/147809, WO/2012/004258, WO/2012/028647 and WO/2012/059549, and WO/2014/144100; and any one or more of the compounds reported by Li et al., Eur J Med Chem 2019; 173: 107-116 (doi: 10.1016/j.ejmech.2019.04.014), or any combination thereof.

In one exemplary, non-limiting embodiment, the sGC activator is selected from any one or more of cinaciguat (BAY58-2667), BAY60-2770, ataciguat (HMR 1766), BI 703704, BI 684067, S-3448, BR-11257, MGV-354, TY-55002, and the compounds claimed in international patent applications WO/2001/19355, WO/2001/19776, WO/2001/19778, WO/2001/19780, WO/2002/070462, WO/2002/070510, and WO/2009/032249, or any combination thereof.

In one embodiment, the pharmaceutical composition is intended for oral administration, optionally designed for delayed or sustained release of its active contents. Oral route of administration of a combination of at least an sGCs and at least an sGCa, or of separate dosage forms of at least an sGCs and at least an sGCa, is preferred.

In one embodiment, the pharmaceutical composition is intended for parenteral administration; preferably, for intravascular administration.

In one embodiment, the pharmaceutical composition is intended for transdermal administration, preferably formulated as a transdermal patch.

In one embodiment, the pharmaceutical composition is intended to treat acute vascular ischemic conditions; including, but not limited to, cerebral or myocardial ischemia, and vaso-occlusive crisis of sickle cell disease.

In one embodiment, the pharmaceutical composition is intended to treat chronic vascular ischemic conditions; including, but not limited to, heart failure with or without preserved ejection fraction, pulmonary arterial hypertension, or disorders of memory, speech and behaviour resulting from impaired cerebral blood flow.

In one embodiment, the pharmaceutical composition is intended to treat male erectile dysfunction. In a preferred embodiment of this application of the invention the pharmaceutical composition is a transdermal gel, cream, or other topical composition.

In one embodiment, the pharmaceutical composition is intended to treat ophthalmological conditions, preferably glaucoma, or conditions associated with retinal degenerative diseases.

In one preferred embodiment of this application of the invention the pharmaceutical composition is an ophthalmologically compatible topical composition, such as an eye-drop.

In another preferred embodiment of this application of the invention the pharmaceutical composition is suitable for intravitreal injection.

Scheme 1: Before the present invention, sGC stimulators and sGC activators were thought to have distinct targets; sGC stimulators stimulate heme containing sGC and augment its stimulation by NO; sGC activators activate apo-sGC, which is heme-free and insensitive to NO. sGC stimulators were considered inactive/irrelevant with respect to apo-sGC.

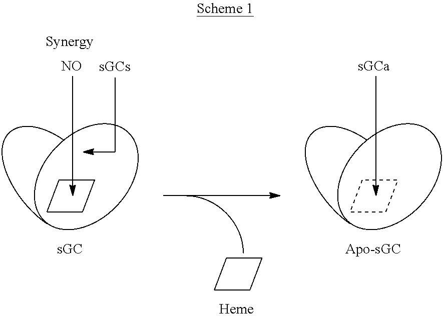

Scheme 2 describes the concept on the use of sGC stimulators and sGC activators according to the present invention. sGC stimulators act on both heme containing sGC (which they directly stimulate and augment its stimulation by NO) and on apo-sGC. In both cases they directly stimulate and augment the stimulation/activation by NO and sGC activators, respectively. Thus the use and suitability in treatment regimens of sGC stimulators is herewith extended to conditions where apo-sGC is present or even increased as a mechanism of disease and to combinations with sGC activators.

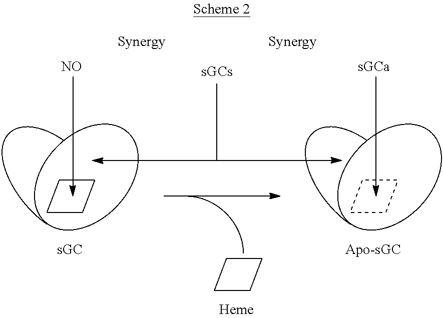

An aspect of the invention relates to a pharmaceutical composition comprising a soluble guanylate cyclase (sGC) stimulator compound (sGCs) and an sGC activator compound (sGCa).

An embodiment is the pharmaceutical composition according to the invention, comprising at least two sGCs and at least one sGCa, or one sGCs and at least two sGCa.

An embodiment is the pharmaceutical composition of the invention, wherein the sGCs is/are any one or more of the sGCs listed in Table 3 and/or the sGCa is/are any one or more of the sGCa listed in Table 4.

An embodiment is the pharmaceutical composition of the invention, wherein the sGCs is/are any one or more of riociguat (BAY 63-2521), vericiguat (BAY 1021189/MK-1242-001), nelociguat (desmethyl riociguat), olinciguat (IW-1701), BAY 41-2272, BAY 60-4552, IWP-953, A-350619, CF-1571, CFM-1571, lificiguat (YC-1), etriciguat, praliciguat (IW-1973) and/or the sGCa is/are any one or more of cinaciguat (BAY 58-2667), BAY 60-2770, ataciguat (HMR 1766), BI 703704, BI 684067, S-3448, BR-11257, MGV-354, TY-55002. Preferred is the pharmaceutical composition of the invention, wherein the sGCs is/are any one or more of riociguat (BAY 63-2521), vericiguat (BAY 1021189/MK-1242-001), nelociguat (desmethyl riociguat), olinciguat (IW-1701), BAY 41-2272, BAY 60-4552, IWP-953, A-350619, CF-1571, CFM-1571, lificiguat (YC-1), etriciguat, praliciguat (IW-1973), preferably one or two of riociguat (BAY 63-2521) and vericiguat (BAY 1021189/MK-1242-001), and/or the sGCa is/are any one or more of cinaciguat (BAY 58-2667), BAY 60-2770, ataciguat (HMR 1766), BI 703704, BI 684067, S-3448, BR-11257, MGV-354, TY-55002 and Bay 12-11163, preferably Bay 12-11163, preferably the sGCs is riociguat and the sGCa is Bay 12-11163 or the sGCs is vericiguat and the sGCa is Bay 12-1116.

TABLE 3
Phase 2 and Phase 3 clinical trials with sGC stimulators

| sGC | | | Study | Oral dose, |
|---|---|---|---|---|
| stimulator | Indication¹⁾ | Phase | name²⁾ | frequency³⁾⁴⁾ |
| Riociguat | PAH | III | PATENT-1 | 1-2.5 mg tid |
| (BAY | CTEPH | III | CHEST-1 | 1-2.5 mg tid |
| 63-2521) | PAH | III | PATENT-2 | 1-2.5 mg tid |
| | CTEPH | III | CHEST-2 | 1-2.5 mg tid |
| | PAH | III | PATENT | 0.5-2.5 |
| | children | | CHILD | mg tid |
| | PAH | III | RESPITE | 0.5-2.5 |
| | | | | mg tid |
| | PH-LVD | II | LEPHT | 0.5-2 mg tid |
| | PH-IIPs | II | RISE IIP⁵⁾ | 0.5-2.5 |
| | | | | mg tid |
| | dcSSc⁶⁾ | II | RISE SSc | 0.5-2.5 |
| | | | | mg tid |
| | CF | II | NCT02170025 | 0.5-2.0 |
| | | | | mg tid |
| | SCD | II | NCT02633397 | 0.5-2.5 |
| | | | | mg tid |
| Nelociguat | ED | II | NCT01168817 | 1.0 mg × 3 |
| (BAY | | | | once daily |
| 60-4552) | | | | |
| Vericiguat | HFrEF | II | SOCRATES- | 1.25-10 mg |
| (BAY 102- | | | REDUCED | once daily |
| 1189) | HFpEF | II | SOCRATES- | 1.25-10 mg |
| | | | PRESERVED | once daily |
| | HFrEF | III | VICTORIA | 2.5-10 mg |
| | | | | once daily |
| | HFpEF | II | VITALITY- | 2.5-15 mg |
| | | | HFpEF | once daily |
| Olinciguat | Achalasia | II | NCT02931565 | 5 mg single dose |
| (IW-1701) | SCD | II | STRONG SCD | Not specified |
| Praliciguat | T2D & | II | NCT03091920 | 40 mg once |
| (IW-1973) | HTN | | | daily/20 |
| | | | | mg twice daily |
| | T2D & | II | NCT02906579 | 10-50 mg |
| | HTN | | | once daily |
| | HFpEF | II | CAPACITY- | Not specified |
| | | | HFpEF | |
| | Diabetic | II | NCT03217591 | Not specified |
| | nephropathy | | | |

¹⁾abbreviations used: PAH, pulmonary arterial hypertension; CTPEH, chronic thromboembolic pulmonary hypertension; PH, pulmonary hypertension; PH-LVD, pulmonary hypertension-left ventricular systolic dysfunction; PH-IIPs, pulmonary hypertension-idiopathic interstitial pneumonias; dcSSc, diffuse cutaneous systemic sclerosis; CF, cystic fibrosis; SCD, sickle cell disease; ED, erectile dysfunction; HFrEF, heart failure with reduced ejection fraction; HFpEF, heart failure with preserved ejection fraction; T2D, type 2 diabetes mellitus; HTN, hypertension; ADHF, acute decompensated chronic congestive heart failure; MCAVS, moderate calcific aortic valve stenosis; PAD, peripheral arterial disease
²⁾information retrieved from publically accessible database ClinicalTrials.gov (clinicaltrials.gov), at 17 Jul. 2019, and information retrieved from the reference book chapter published online as “Soluble Guanylate Cyclase Stimulators and Activators”, Sandner, P., Zimmer, D.P., Todd Milne, G., Follmann, M., Hobbs, A., Stasch, J-.P., Handbook of Experimental Pharmacology, 2018, pp.1-40, Springer Nature Switzerland AG, doi number 10.1007/164_2018_197, first online at 29 Jan. 2019.
³⁾As 2), and in addition, information is retrieved from published papers, etc., reporting on the listed phase II and III clinical trials.
⁴⁾Abbreviations used: tid, three times daily; OR, orally
⁵⁾the study is terminated due to adverse events.
⁶⁾Bayer/MSD co-development; BAY 102-1189 = MK-1242-001

An embodiment is the pharmaceutical composition of the invention, wherein the sGCs is at least one of riociguat (BAY 63-2521), nelociguat (BAY 60-4552), vericiguat (BAY 102-1189), olinciguat (IW-1701), praliciguat (IW-1973), and wherein the sGCa is ataciguat (HMR 1766) or Bay 12-1116.

TABLE 4
Phase 2 clinical trials with sGC activators

| | | | | Dose, frequency |
|---|---|---|---|---|
| sGC | | | | (route of |
| activator | Indication¹⁾ | Phase | Study name²⁾ | administration)³⁾⁴⁾ |
| Cinaciguat | ADHF | II | COMPOSE | 50, 100, |
| (BAY 58- | | | 1⁵⁾ | 150 μg/h (IV) |
| 2667) | ADHF | II | NCT00559650⁶⁾ | 100-600 |
| | | | | μg/g (IV) |
| | ADHF | IIb | COMPOSE 2⁵⁾ | 10, 25 |
| | | | | μg/h (IV) |
| | ADHF | II | COMPOSE | 50, 100, 150 |
| | | | EARLY¹ | μg/h (IV) |
| Ataciguat | MCAVS | I | NCT02049203 | 50, 100 or 200 |
| (HMR | | | | mg once daily |
| 1766) | | | | (OR) |
| | PAD | II | ACCELA | Not specified |
| | | | | (OR) |
| | MCAVS | II | NCT02481258 | 200 mg once |
| | | | | daily (OR) |
| | Neuropathic | II | SERENEATI | 200 mg once |
| | pain | | | daily (OR) |

¹⁾abbreviations used: ADHF, acute decompensated chronic congestive heart failure; MCAVS, moderate calcific aortic valve stenosis; PAD, peripheral arterial disease
²⁾information retrieved from publically accessible database ClinicalTrials.gov (clinicaltrials.gov), at 17 Jul. 2019, and information retrieved from the reference book chapter published online as “Soluble Guanylate Cyclase Stimulators and Activators”, Sandner, P., Zimmer, D.P., Todd Milne, G., Follmann, M., Hobbs, A., Stasch, J-.P., Handbook of Experimental Pharmacology, 2018, pp.1-40, Springer Nature Switzerland AG, doi number 10.1007/164_2018_197, first online at 29 Jan. 2019.
³⁾As 2), and in addition, if present, information is retrieved from published papers, etc., reporting on the listed phase II clinical trials.
⁴⁾Abbreviations used: IV, intravenously; OR, orally
⁵⁾terminated due to lack of efficacy combined with adverse event (hypotension)
⁶⁾terminated due to adverse events at high doses > 200 (hypotension)

An embodiment is the pharmaceutical composition of the invention, wherein the sGCs is provided as a unit dose comprising 0.05 mg-100 mg of the one or more sGCs, such as 0.1 mg-50 mg, preferably 0.2 mg-25 mg, more preferably 0.4 mg-10 mg, most preferably 1 mg-5 mg, such as 2 mg-3 mg, and/or wherein the sGCa is provided as a unit dose comprising 0.5 mg-500 mg of the one or more sGCa or ataciguat when depending on claim 5, preferably 1 mg-300 mg, more preferably 2 mg-200 mg, most preferably 3 mg-100 mg, such as 4 mg-50 mg, or 5 mg-25 mg.

An embodiment is the pharmaceutical composition of the invention, wherein the pharmaceutical composition further comprises a further active pharmaceutical ingredient.

An embodiment is the pharmaceutical composition of the invention, wherein the sGCs is provided as a unit dose comprising 0.1 mg-5 mg riociguat, preferably less than 2.5 mg, 0.05 mg-2 mg nelociguat, preferably less than 1 mg, 0.1 mg-30 mg vericiguat, preferably less than 15 mg, 0.1 mg-10 mg olinciguat, preferably less than 5 mg, 1 mg-100 mg praliciguat, preferably less than 50 mg, and/or wherein the sGCa is provided as a unit dose comprising 5 mg-400 mg ataciguat, preferably less than 200 mg.

An embodiment is the pharmaceutical composition according to the invention, further comprising at least one nitric-oxide (NO) donor compound.

An embodiment is the pharmaceutical composition according to the invention, wherein the at least one NO donor compound is any one or more compound(s) selected from an organic nitrate, an organic nitrite, an S-nitrosothiol, a prusside, an NONOate, a sydnonimine, an oxatriazole, a furoxan, a Ruthenium nitrosyl, a photochemical donor via one/two-photon excitation, a diazeniumdiolated carbamate, nitroglycerin, molsidomine, isosorbide dinitrate (ISDN), sodium nitroprusside or an alternative pharmaceutically acceptable nitroprusside salt, and any pharmaceutically acceptable derivative thereof and/or any pharmaceutically acceptable salt thereof and/or any pharmaceutically acceptable prodrug thereof.

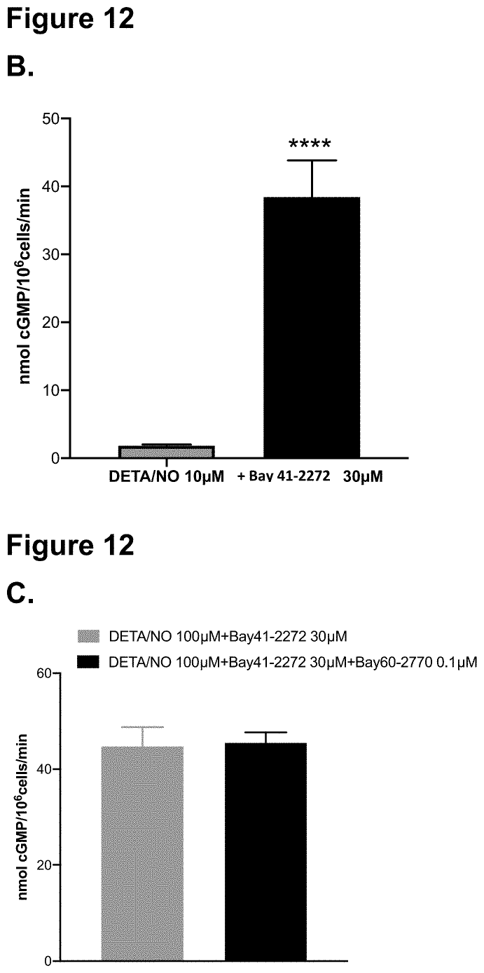

As exemplified in the Example section here below, and in particular in FIG. 12, the inventors surprisingly found that cGMP production by cells synergistically increased when those cells had apo-sGC and were stimulated with a combination of an NO donor compound (here, DETA-NONOate) and an sGC stimulator (here, Bay41-2272). The cells hardly produced any cGMP upon stimulation of the apo-sGC with the NO donor compound only. Stimulation of the apo-sGC with the sGC stimulator resulted in cGMP production by the cells. Combining the sGC stimulator with the NO donor compound resulted in a more than double amount of cGMP, demonstrating the synergistic manner in which the sGC stimulator compound and the NO donor compound are able to activate the apo-sGC.

An aspect of the invention is an oral dose combination comprising a first oral dose comprising an sGCs and a second oral dose comprising an sGCa, the first oral dose and the second oral dose optionally comprising one or more pharmaceutically acceptable excipient(s).

An embodiment is the oral dose combination of the invention, wherein the sGCs is an sGCs according to any of the previous aspects and embodiments and/or wherein the sGCs is an sGCs comprised by the oral dose combination of the invention provided at the dose of the invention, and/or wherein the sGCa is an sGCa according to any of the previous aspects and embodiments and/or wherein the sGCa is an sGCa comprised by the oral dose combination of the invention provided at the dose of the invention.

An embodiment is the oral dose combination of the invention, further comprising a third oral dose comprising an NO donor compound, preferably an NO donor compound selected from an organic nitrate, an organic nitrite, an S-nitrosothiol, a prusside, an NONOate, a sydnonimine, an oxatriazole, a furoxan, a Ruthenium nitrosyl, a photochemical donor via one/two-photon excitation, a diazeniumdiolated carbamate, nitroglycerin, molsidomine, isosorbide dinitrate (ISDN), sodium nitroprusside or an alternative pharmaceutically acceptable nitroprusside salt, and any pharmaceutically acceptable derivative thereof and/or any pharmaceutically acceptable salt thereof and/or any pharmaceutically acceptable prodrug thereof.

An embodiment is the pharmaceutical composition of the invention, wherein the sGCs and the sGCa are provided as a solid dosage form such as a capsule or a tablet, and wherein the NO donor compound, if present, is provided as a solid dosage form, or oral dose combination of the invention, wherein the sGCs and the sGCa each are provided separately as a solid dosage form such as a capsule or a tablet, and when present, wherein the NO donor compound is provided separately from the sGCs and the sGCa as a solid dosage form.

An embodiment is the pharmaceutical composition of the invention wherein a single unit of the solid dosage form contains a daily dosage of the one or more sGCs and the one or more sGCa and if present the one or more NO donor compound(s), or oral dose combination of the invention, wherein a single unit of the solid dosage form containing the sGCs contains a daily dosage of the one or more sGCs and/or a single unit of the solid dosage form containing the sGCa contains a daily dosage of the one or more sGCa and if present the one or more NO donor compound(s).

Again, as exemplified in the Example section here below, and in particular in FIG. 12, the inventors surprisingly found that cGMP production by cells synergistically increased when those cells had apo-sGC and were stimulated with a combination of an NO donor compound (here, DETA-NONOate) and an sGC stimulator (here, Bay41-2272). The cells hardly produced any cGMP upon stimulation of the apo-sGC with the NO donor compound only. Stimulation of the apo-sGC with the sGC stimulator resulted in cGMP production by the cells. Combining the sGC stimulator with the NO donor compound resulted in a more than double amount of cGMP, demonstrating the synergistic manner in which the sGC stimulator compound and the NO donor compound are able to activate the apo-sGC.

An embodiment is the pharmaceutical composition of the invention wherein the sGCs and the sGCa are provided as a solid dosage form such as a capsule or a tablet, or oral dose combination of the invention, wherein the sGCs and the sGCa each are provided separately as a solid dosage form such as a capsule or a tablet.

An embodiment is the pharmaceutical composition of the invention wherein a single unit of the solid dosage form contains a daily dosage of the one or more sGCs and the one or more sGCa, or oral dose combination of the invention, wherein a single unit of the solid dosage form containing the sGCs contains a daily dosage of the one or more sGCs and/or a single unit of the solid dosage form containing the sGCa contains a daily dosage of the one or more sGCa.

An aspect of the invention relates to a kit comprising the pharmaceutical composition of the invention or the oral dose combination of the invention, and optionally instructions for use.

An aspect of the invention relates to the pharmaceutical composition of the invention or oral dose combination of the invention, for use as a medicament.

An aspect of the invention relates to the pharmaceutical composition or oral dose combination for use in a method for the treatment of cyclic 3′,5′-guanosine monophosphate (cGMP) deficiency in a patient, preferably a human patient.

An embodiment is the pharmaceutical composition or oral dose combination for use according to the invention, wherein the use is in a method for the treatment of a cardiovascular disease, or wherein the patient deficient in cGMP suffers from a cardiovascular disease.

An embodiment is the pharmaceutical composition or oral dose combination for use according to the invention, wherein the patient to whom the pharmaceutical composition or the oral dose combination is administered, suffers from any one or more of pulmonary arterial hypertension, chronic thromboembolic pulmonary hypertension, pulmonary hypertension, persistent pulmonary hypertension of the new born, portal hypertension, pulmonary hypertension—left ventricular systolic dysfunction, pulmonary hypertension—idiopathic interstitial pneumonias, diffuse cutaneous systemic sclerosis, cystic fibrosis, moyamoya syndrome, sickle cell disease, erectile dysfunction, heart failure with reduced ejection fraction, heart failure with preserved ejection fraction, type 2 diabetes mellitus, hypertension, acute decompensated chronic congestive heart failure, moderate calcific aortic valve stenosis, peripheral arterial disease, fibrotic conditions such as liver fibrosis, NASH, complications relating to diabetes mellitus, such as diabetic nephropathy and diabetic cardiomyopathy, and COVID19-related respiratory distress and/or cardiovascular complications. Preferred is the pharmaceutical composition or oral dose combination for use according to the invention, wherein the patient to whom the pharmaceutical composition or the oral dose combination is administered, suffers from any one or more of pulmonary arterial hypertension, chronic thromboembolic pulmonary hypertension, pulmonary hypertension, persistent pulmonary hypertension of the new born, portal hypertension, pulmonary hypertension—left ventricular systolic dysfunction, pulmonary hypertension—idiopathic interstitial pneumonias, diffuse cutaneous systemic sclerosis, cystic fibrosis, sickle cell disease, erectile dysfunction, heart failure with reduced ejection fraction, heart failure with preserved ejection fraction, type 2 diabetes mellitus, hypertension, acute decompensated chronic congestive heart failure, moderate calcific aortic valve stenosis, peripheral arterial disease, fibrotic conditions such as liver fibrosis, NASH, complications relating to diabetes mellitus, such as diabetic nephropathy and diabetic cardiomyopathy, and COVID19-related respiratory distress and/or cardiovascular complications, ischemia, neonatal asphyxia, oxidative organ damage, oxidative tissue damage, oxidative cell damage, stroke, acute respiratory distress syndrome and asthma, preferably ischemia, oxidative organ damage, oxidative tissue damage, oxidative cell damage and stroke, more preferably ischemia and stroke.

An embodiment is the pharmaceutical composition or oral dose combination for use according to the invention, wherein the pharmaceutical composition is administered to the patient as a single unit dose daily, or as two-four unit doses daily, such as thrice daily, or wherein the first oral dose and/or the second oral dose of the oral dose combination is administered to the patient as a single solid dosage daily, or as two-four solid dosages daily, such as thrice daily.

An embodiment is the pharmaceutical composition or oral dose combination for use according to the invention, wherein the patient in need thereof is administered an effective dose of the pharmaceutical composition of the invention or is administered an effective dose of the oral dose combination of the invention.

An embodiment is the pharmaceutical composition or oral dose combination for use according to the invention, wherein the patient suffers from a disease or disorder accompanied by the presence of apo-sGC, such as any one or more of ischemia, oxidative organ damage, oxidative tissue damage, oxidative cell damage, stroke, acute respiratory distress syndrome, neonatal asphyxia and asthma, preferably ischemia, oxidative organ damage, oxidative tissue damage, oxidative cell damage and stroke, more preferably ischemia and stroke, and optionally wherein the patient suffers from a disease or disorder accompanied by the presence of apo-sGC and the absence of sGC.

An embodiment is the pharmaceutical composition or oral dose combination for use according to the invention, wherein the treatment comprises stimulation of cGMP formation in the patient in need of said treatment.

An embodiment is the pharmaceutical composition or oral dose combination for use according to the invention, wherein the patient suffers from nitric oxide (NO) insufficiency and/or from oxidative damage. Preferred is the pharmaceutical composition or oral dose combination for use according to the invention, wherein the patient suffers from nitric oxide (NO) insufficiency and/or from any one or more of oxidative damage, ischemia, neonatal asphyxia, oxidative organ damage, oxidative tissue damage, oxidative cell damage, stroke, acute respiratory distress syndrome and asthma, preferably from any one or more of nitric oxide (NO) insufficiency and/or ischemia, oxidative organ damage, oxidative tissue damage, oxidative cell damage and stroke, more preferably from nitric oxide (NO) insufficiency and/or ischemia and/or stroke.

An embodiment is the pharmaceutical composition or oral dose combination for use according to the invention, wherein the patient suffers from a medical condition relating to sGC dysfunction and/or relating to cGMP deficiency.

An embodiment is the pharmaceutical composition or oral dose combination for use according to the invention, wherein the sGCs augment(s) stimulation of heme containing sGC and augment(s) stimulation of sGC by NO and/or stimulate(s) apo-sGC.

An embodiment is the pharmaceutical composition or oral dose combination for use according to the invention, wherein the one or more sGCs and the one or more sGCa augment sGC and/or apo-sGC synergistically.

An aspect of the invention relates to a pharmaceutical composition comprising an sGCs for use as a medicament.

An aspect of the invention relates to a pharmaceutical composition comprising at least two sGCs for use as a medicament.

An embodiment is the pharmaceutical composition for use of the invention, wherein the sGCs is an sGCs listed in Table 3 or wherein the sGCs are any two or more of the sGCs listed in Table 3.

An embodiment is the pharmaceutical composition for use of the invention, wherein the sGCs is/are any one or any two or more of riociguat (BAY 63-2521), vericiguat (BAY 1021189/MK-1242-001), nelociguat (desmethyl riociguat), olinciguat (IW-1701), BAY 41-2272, BAY 60-4552, IWP-953, A-350619, CF-1571, CFM-1571, lificiguat (YC-1), etriciguat, praliciguat (IW-1973), preferably one or two of riociguat (BAY 63-2521) and vericiguat (BAY 1021189/MK-1242-001).

An embodiment is the pharmaceutical composition for use of the invention, wherein the sGCs is/are one or at least two of riociguat (BAY 63-2521), nelociguat (BAY 60-4552), vericiguat (BAY 102-1189), olinciguat (IW-1701), praliciguat (IW-1973).

An embodiment is the pharmaceutical composition for use of the invention, wherein the sGCs is/are provided as a unit dose comprising 0.05 mg-100 mg of the one or two or more sGCs, such as 0.1 mg-50 mg, preferably 0.2 mg-25 mg, more preferably 0.4 mg-10 mg, most preferably 1 mg-5 mg, such as 2 mg-3 mg.

An embodiment is the pharmaceutical composition for use of the invention, wherein the pharmaceutical composition further comprises a further active pharmaceutical ingredient.

An embodiment is the pharmaceutical composition for use of the invention, wherein the sGCs is/are provided as a unit dose comprising any one or any two or more of 0.1 mg-5 mg riociguat, preferably less than 2.5 mg, 0.05 mg-2 mg nelociguat, preferably less than 1 mg, 0.1 mg-30 mg vericiguat, preferably less than 15 mg, 0.1 mg-10 mg olinciguat, preferably less than 5 mg, 1 mg-100 mg praliciguat, preferably less than 50 mg.

An embodiment is the pharmaceutical composition for use of the invention, wherein the pharmaceutical composition further comprises at least one nitric-oxide (NO) donor compound.

An embodiment is the pharmaceutical composition for use according to the invention, wherein the at least one NO donor compound is any one or more compound(s) selected from an organic nitrate, an organic nitrite, an S-nitrosothiol, a prusside, an NONOate, a sydnonimine, an oxatriazole, a furoxan, a Ruthenium nitrosyl, a photochemical donor via one/two-photon excitation, a diazeniumdiolated carbamate, nitroglycerin, molsidomine, isosorbide dinitrate (ISDN), sodium nitroprusside or an alternative pharmaceutically acceptable nitroprusside salt, and any pharmaceutically acceptable derivative thereof and/or any pharmaceutically acceptable salt thereof and/or any pharmaceutically acceptable prodrug thereof.

An aspect of the invention relates to an oral dose combination comprising a first oral dose comprising a first sGCs and a second oral dose comprising a second sGCs, the first oral dose and the second oral dose optionally comprising one or more pharmaceutically acceptable excipient(s), for use according to the invention.

An embodiment is the oral dose combination for use of the invention, wherein the first sGCs is an sGCs according to the invention and/or wherein the first sGCs is an sGCs of the oral dose combination of the invention provided at the dose of the invention, and/or wherein the second sGCs is a second sGCs according to the invention and different from the first sGCs and/or wherein the second sGCs is an sGCs of the oral dose combination of the invention different from the first sGCs provided at the dose of the invention.

An embodiment is the pharmaceutical composition for use of the invention wherein the sGCs is/are provided as a solid dosage form such as a capsule or a tablet, or oral dose combination for use of the invention, wherein the first sGCs and the second sGCs each are provided separately as a solid dosage form such as a capsule or a tablet.

An embodiment is the oral dose combination of the invention, further comprising a third oral dose comprising an NO donor compound, preferably an NO donor compound selected from an organic nitrate, an organic nitrite, an S-nitrosothiol, a prusside, an NONOate, a sydnonimine, an oxatriazole, a furoxan, a Ruthenium nitrosyl, a photochemical donor via one/two-photon excitation, a diazeniumdiolated carbamate, nitroglycerin, molsidomine, isosorbide dinitrate (ISDN), sodium nitroprusside or an alternative pharmaceutically acceptable nitroprusside salt, and any pharmaceutically acceptable derivative thereof and/or any pharmaceutically acceptable salt thereof and/or any pharmaceutically acceptable prodrug thereof.

An embodiment is the oral dose combination comprising a first oral dose comprising an sGCs and a second oral dose comprising an NO donor compound, preferably an NO donor compound according to the invention, the first oral dose and the second oral dose optionally comprising one or more pharmaceutically acceptable excipient(s), for use according to the invention.

An embodiment is the pharmaceutical composition for use of the invention wherein the sGCs is/are provided as a solid dosage form such as a capsule or a tablet, and if present, wherein the at least one NO donor compound is provided separately as a solid dosage form, or oral dose combination for use of the invention, wherein the first sGCs and the second sGCs each are provided separately as a solid dosage form such as a capsule or a tablet, and when present, wherein the at least one NO donor compound is provided separately as a solid dosage form.

An embodiment is the pharmaceutical composition for use of the invention wherein a single unit of the solid dosage form contains a daily dosage of the one or the two or more sGCs, or oral dose combination for use of the invention, wherein a single unit of the solid dosage form containing the first sGCs contains a daily dosage of the first sGCs and/or a single unit of the solid dosage form containing the second sGCs contains a daily dosage of the second sGCs, and if present, wherein a single unit of the solid dosage form containing the NO donor compound contains a daily dosage of the NO donor compound.

As exemplified in the Example section here below, and in particular in FIG. 12, the inventors surprisingly found that cGMP production by cells synergistically increased when those cells had apo-sGC and were stimulated with a combination of an NO donor compound (here, DETA-NONOate) and an sGC stimulator (here, Bay41-2272). The cells hardly produced any cGMP upon stimulation of the apo-sGC with the NO donor compound only. Stimulation of the apo-sGC with the sGC stimulator resulted in cGMP production by the cells. Combining the sGC stimulator with the NO donor compound resulted in a more than double amount of cGMP, demonstrating the synergistic manner in which the sGC stimulator compound and the NO donor compound are able to activate the apo-sGC.

An aspect of the invention relates to a pharmaceutical composition comprising a soluble guanylate cyclase (sGC) stimulator compound (sGCs) and an sGC activator compound (sGCa), or oral dose combination comprising a first oral dose comprising an sGCs and a second oral dose comprising an sGCa, the first oral dose and the second oral dose optionally comprising one or more pharmaceutically acceptable excipient(s), for use as a medicament.

An aspect of the invention relates to a pharmaceutical composition comprising a soluble guanylate cyclase (sGC) stimulator compound (sGCs) and an sGC activator compound (sGCa), or oral dose combination comprising a first oral dose comprising an sGCs and a second oral dose comprising an sGCa, the first oral dose and the second oral dose optionally comprising one or more pharmaceutically acceptable excipient(s), for use in a method for the treatment of cyclic 3′5′-guanosine monophosphate (cGMP) deficiency in a patient, preferably a human patient, wherein the patient suffers from a disease or disorder accompanied by the presence of apo-sGC, such as any one or more of ischemia, oxidative organ damage, oxidative tissue damage, oxidative cell damage, stroke, acute respiratory distress syndrome, neonatal asphyxia and asthma, preferably ischemia, neonatal asphyxia, oxidative organ damage, oxidative tissue damage, oxidative cell damage and stroke, more preferably ischemia and stroke, and optionally wherein the patient suffers from a disease or disorder accompanied by the presence of apo-sGC and the absence of sGC.

Optionally, the pharmaceutical composition for use, or the oral dose combination for use according to the invention comprises at least two sGCs and at least one sGCa, or one sGCs and at least two sGCa.

Preferred is the pharmaceutical composition for use of the invention or the oral dose combination for use of the invention, wherein the sGCs is/are any one or more of the sGCs listed in Table 3 and/or the sGCa is/are any one or more of the sGCa listed in Table 4.

Typically, in the pharmaceutical composition for use of the invention or in the oral dose combination for use of the invention, the sGCs is/are any one or more of riociguat (BAY 63-2521), vericiguat (BAY 1021189/MK-1242-001), nelociguat (desmethyl riociguat), olinciguat (IW-1701), BAY 41-2272, BAY 60-4552, BAY 63-2521, IWP-953, A-350619, CF-1571, CFM-1571, lificiguat (YC-1), etriciguat, praliciguat (IW-1973), preferably one or two of riociguat (BAY 63-2521) and vericiguat (BAY 1021189/MK-1242-001), and/or the sGCa is/are any one or more of cinaciguat (BAY 58-2667), BAY 60-2770, ataciguat (HMR 1766), BI 703704, BI 684067, S-3448, BR-11257, MGV-354, TY-55002 and Bay 12-11163, preferably Bay 12-11163, preferably the sGCs is riociguat and the sGCa is Bay 12-11163 or the sGCs is vericiguat and the sGCa is Bay 12-1116.

A preferred pharmaceutical composition for use according to the invention or a preferred oral dose combination for use according to the invention, is a pharmaceutical composition, wherein the sGCs is at least one of riociguat (BAY 63-2521), nelociguat (BAY 60-4552), vericiguat (BAY 102-1189), olinciguat (IW-1701), praliciguat (IW-1973), and wherein the sGCa is ataciguat (HMR 1766) or Bay 12-1116.

Typically, for the pharmaceutical composition for use of the invention, the sGCs is provided as a unit dose comprising 0.05 mg-100 mg of the one or more sGCs, such as 0.1 mg-50 mg, preferably 0.2 mg-25 mg, more preferably 0.4 mg-10 mg, most preferably 1 mg-5 mg, such as 2 mg-3 mg, and/or the sGCa is provided as a unit dose comprising 0.5 mg-500 mg of the one or more sGCa or of ataciguat, preferably 1 mg-300 mg, more preferably 2 mg-200 mg, most preferably 3 mg-100 mg, such as 4 mg-50 mg, or 5 mg-25 mg.

Optional is the pharmaceutical composition for use of the invention, wherein the pharmaceutical composition further comprises a further active pharmaceutical ingredient.

Preferred is a pharmaceutical composition for use of the invention, wherein the sGCs is provided as a unit dose comprising 0.1 mg-5 mg riociguat, preferably less than 2.5 mg, 0.05 mg-2 mg nelociguat, preferably less than 1 mg, 0.1 mg-30 mg vericiguat, preferably less than 15 mg, 0.1 mg-10 mg olinciguat, preferably less than 5 mg, 1 mg-100 mg praliciguat, preferably less than 50 mg, and/or wherein the sGCa is provided as a unit dose comprising 5 mg-400 mg ataciguat, preferably less than 200 mg.

Also preferred is the pharmaceutical composition for use according to the invention or the oral dose combination for use of the invention, the pharmaceutical composition or the oral dose combination further comprising at least one nitric-oxide (NO) donor compound.

Typically, for the pharmaceutical composition for use according to the invention or for the oral dose combination for use according to the invention, the at least one NO donor compound is any one or more compound(s) selected from an organic nitrate, an organic nitrite, an S-nitrosothiol, a prusside, an NONOate, a sydnonimine, an oxatriazole, a furoxan, a Ruthenium nitrosyl, a photochemical donor via one/two-photon excitation, a diazeniumdiolated carbamate, nitroglycerin, molsidomine, isosorbide dinitrate (ISDN), sodium nitroprusside or an alternative pharmaceutically acceptable nitroprusside salt, and any pharmaceutically acceptable derivative thereof and/or any pharmaceutically acceptable salt thereof and/or any pharmaceutically acceptable prodrug thereof.

An embodiment is the pharmaceutical composition for use of the invention wherein the sGCs and the sGCa are provided as a solid dosage form such as a capsule or a tablet, and when applicable, wherein the NO donor compound is provided as a solid dosage form, or an embodiment is the oral dose combination for use of the invention, wherein the sGCs and the sGCa each are provided separately as a solid dosage form such as a capsule or a tablet, and when applicable, wherein the NO donor compound is provided separately from the sGCs and the sGCa as a solid dosage form.

Optional is the pharmaceutical composition for use according to the invention, wherein a single unit of the solid dosage form contains a daily dosage of the one or more sGCs and the one or more sGCa and if present the one or more NO donor compound(s), or optional is the oral dose combination for use of the invention, wherein a single unit of the solid dosage form containing the sGCs contains a daily dosage of the one or more sGCs and/or a single unit of the solid dosage form containing the sGCa contains a daily dosage of the one or more sGCa and if present the one or more NO donor compound(s).

An aspect of the invention relates to a kit comprising the pharmaceutical composition for use of the invention or a kit comprising the oral dose combination for use of the invention, and optionally instructions for use.

Preferred is the pharmaceutical composition for use of the invention or the oral dose combination for use according to the invention, wherein the use is in a method for the treatment of a cardiovascular disease, or wherein the patient deficient in cGMP suffers from a cardiovascular disease.

Also preferred is the pharmaceutical composition for use of the invention or the oral dose combination for use according to the invention, wherein the patient to whom the pharmaceutical composition or the oral dose combination is administered, suffers from any one or more of pulmonary arterial hypertension, chronic thromboembolic pulmonary hypertension, pulmonary hypertension, persistent pulmonary hypertension of the new born, portal hypertension, pulmonary hypertension—left ventricular systolic dysfunction, pulmonary hypertension—idiopathic interstitial pneumonias, diffuse cutaneous systemic sclerosis, cystic fibrosis, sickle cell disease, erectile dysfunction, heart failure with reduced ejection fraction, heart failure with preserved ejection fraction, type 2 diabetes mellitus, hypertension, acute decompensated chronic congestive heart failure, moderate calcific aortic valve stenosis, peripheral arterial disease, ischemia, neonatal asphyxia, oxidative organ damage, oxidative tissue damage, oxidative cell damage, stroke, acute respiratory distress syndrome and asthma, preferably ischemia, oxidative organ damage, oxidative tissue damage, oxidative cell damage and stroke, more preferably ischemia and stroke.

An embodiment is the pharmaceutical composition for use of the invention wherein a single unit of the solid dosage form contains a daily dosage of the one or the two or more sGCs, or oral dose combination for use of the invention, wherein a single unit of the solid dosage form containing the first sGCs contains a daily dosage of the first sGCs and/or a single unit of the solid dosage form containing the second sGCs contains a daily dosage of the second sGCs.

An embodiment is the pharmaceutical composition or oral dose combination for use according to the invention, wherein the use is in a method for the treatment of cyclic 3′,5′-guanosine monophosphate (cGMP) deficiency in a patient, preferably a human patient.

An embodiment is the pharmaceutical composition or oral dose combination for use according to the invention, wherein the use is in a method for the treatment of a cardiovascular disease, or wherein the patient deficient in cGMP suffers from a cardiovascular disease.

An embodiment is the pharmaceutical composition or oral dose combination for use according to the invention, wherein the patient to whom the pharmaceutical composition or the oral dose combination is administered, suffers from any one or more of pulmonary arterial hypertension, chronic thromboembolic pulmonary hypertension, pulmonary hypertension, persistent pulmonary hypertension of the new born, portal hypertension, pulmonary hypertension—left ventricular systolic dysfunction, pulmonary hypertension—idiopathic interstitial pneumonias, diffuse cutaneous systemic sclerosis, cystic fibrosis, moyamoya syndrome, sickle cell disease, erectile dysfunction, heart failure with reduced ejection fraction, heart failure with preserved ejection fraction, type 2 diabetes mellitus, hypertension, acute decompensated chronic congestive heart failure, moderate calcific aortic valve stenosis, peripheral arterial disease, erectile dysfunction, fibrotic conditions such as liver fibrosis, NASH, complications relating to diabetes mellitus, such as diabetic nephropathy and diabetic cardiomyopathy, and COVID19-related respiratory distress and/or cardiovascular complications, ischemia, neonatal asphyxia, oxidative organ damage, oxidative tissue damage, oxidative cell damage, stroke, acute respiratory distress syndrome and asthma, preferably ischemia, neonatal asphyxia, oxidative organ damage, oxidative tissue damage, oxidative cell damage and stroke, more preferably ischemia and stroke. An embodiment is the pharmaceutical composition or oral dose combination for use according to the invention, wherein the pharmaceutical composition is administered to the patient as a single unit dose daily, or as two-four unit doses daily, such as thrice daily, or wherein the first oral dose and/or the second oral dose of the oral dose combination is administered to the patient as a single solid dosage daily, or as two-four solid dosages daily, such as thrice daily.

An embodiment is the pharmaceutical composition or oral dose combination for use according to the invention, wherein the patient in need thereof is administered an effective dose of the pharmaceutical composition of the invention or is administered an effective dose of the oral dose combination of the invention.

An embodiment is the pharmaceutical composition or oral dose combination for use according to the invention, wherein the patient suffers from a disease or disorder accompanied by the presence of apo-sGC. Preferred is the pharmaceutical composition or oral dose combination for use according to the invention, wherein the patient suffers from a disease or disorder accompanied by the presence of apo-sGC, such as any one or more of ischemia, neonatal asphyxia, oxidative organ damage, oxidative tissue damage, oxidative cell damage, stroke, acute respiratory distress syndrome and asthma, preferably ischemia, neonatal asphyxia, oxidative organ damage, oxidative tissue damage, oxidative cell damage and stroke, more preferably ischemia and stroke, and optionally wherein the patient suffers from a disease or disorder accompanied by the presence of apo-sGC and the absence of sGC.

An embodiment is the pharmaceutical composition or oral dose combination for use according to the invention, wherein the treatment comprises stimulation of cGMP formation in the patient in need of said treatment.

An embodiment is the pharmaceutical composition or oral dose combination for use according to the invention, wherein the patient suffers from nitric oxide (NO) insufficiency and/or from oxidative damage. Preferred is the pharmaceutical composition or oral dose combination for use according to the invention, wherein the patient suffers from nitric oxide (NO) insufficiency and/or from any one or more of oxidative damage, ischemia, neonatal asphyxia, oxidative organ damage, oxidative tissue damage, oxidative cell damage, stroke, acute respiratory distress syndrome and asthma, preferably from any one or more of nitric oxide (NO) insufficiency and/or ischemia, neonatal asphyxia, oxidative organ damage, oxidative tissue damage, oxidative cell damage and stroke, more preferably from nitric oxide (NO) insufficiency and/or ischemia and/or stroke.

An embodiment is the pharmaceutical composition or oral dose combination for use according to the invention, wherein the patient suffers from a medical condition relating to sGC dysfunction and/or relating to cGMP deficiency.

An embodiment is the pharmaceutical composition or oral dose combination for use according to the invention, wherein the sGCs augment(s) stimulation of heme containing sGC and augment(s) stimulation of sGC by NO and/or stimulate(s) apo-sGC.

An embodiment is the pharmaceutical composition or oral dose combination for use according to the invention, wherein the two or more sGCs augment sGC and/or apo-sGC synergistically.

An aspect of the invention relates to the use of an sGCs and an sGCa for the manufacture of a medicament for the treatment of cGMP deficiency.

An embodiment is the use of an sGCs and an sGCa for the manufacture of the invention, wherein the medicament is for the treatment of a CVD.

An aspect of the invention relates to a method for treating a human subject with an sGCs, a combination of two sGCs or a combination of at least an sGCs and at least an sGCs, wherein the human subject is suffering from a cardiovascular disease and/or form cGMP deficiency, the method comprising the steps of: determining the level of cGMP in said human subject by: 1) obtaining or having obtained a blood sample from the human subject; 2) performing or having performed a cGMP concentration determining assay on cells derived from the blood sample to determine if the patient is deficient in cGMP; and 3) if the human subject is deficient in cGMP then internally administering sGCs or sGCs and sGCa to the patient

While the invention has been described with respect to various embodiments, such embodiments are not limiting. Numerous variations and modifications would be understood by those of ordinary skill in the art. Such variations and modifications are considered to be included within the scope of the claims.

The present invention has been described above with reference to a number of exemplary embodiments as shown in the drawings. Modifications and alternative implementations of some parts or elements are possible, and are included in the scope of protection as defined in the appended claims.

Typically, the one or more sGCs compounds comprised by the pharmaceutical composition of the invention, the dose combination such as an oral dose combination of the invention, and the one or more sGCs compounds comprised by the pharmaceutical composition of the invention or comprised by the dose combination for use as a medicament or for use in a method for the treatment of any one of the indicated diseases, health problems, disorders, deficiencies, etc., according to the invention, are selected from the tabulated sGCs compounds in Table 2 or Table 3. Typically, the one or more sGCa compounds comprised by the pharmaceutical composition of the invention, the dose combination such as an oral dose combination of the invention, and the one or more sGCa compounds comprised by the pharmaceutical composition of the invention or comprised by the dose combination for use as a medicament or for use in a method for the treatment of any one of the indicated diseases, health problems, disorders, deficiencies, etc., according to the invention, are selected from the tabulated sGCa compounds in Table 2 or Table 4.

Embodiments are the pharmaceutical composition of the invention, the dose combination such as an oral dose combination of the invention and any of the pharmaceutical compositions or any of the dose combinations for use according to the invention, wherein the sGCs is/are selected from the sGCs listed in Table 2 or Table 3, and wherein the sGCa is/are selected from the sGCa listed in Table 2 or Table 4.

Preferred are the pharmaceutical compositions or any of the dose combinations for use according to the invention, wherein the patient in need of treatment with such a pharmaceutical composition or such a dose combination suffers from any of the diseases, CVDs, disorders tabulated in Table 3 or in Table 4. Typically, the pharmaceutical compositions or any of the dose combinations for use according to the invention, are used in a method for the treatment of a patient in need of treatment with such a pharmaceutical composition or such a dose combination, wherein the patient suffers from any of the diseases, CVDs, disorders tabulated in Table 4. For example, the patient in need of such treatment suffers from acute decompensated chronic congestive heart failure, moderate calcific aortic valve stenosis, and/or peripheral arterial disease.

Embodiments are the pharmaceutical compositions or any of the dose combinations for use according to the invention, wherein the patient in need of treatment with such a pharmaceutical composition or such a dose combination suffers from any of the diseases, CVDs, disorders cGMP deficiency, stroke, hypoxia, heart infarct, oxidative damage, post-reperfusion oxidative damage, (abundant) presence of apo-sGC, (risk for) hypotension, NO-insensitive sGC. Alternatively, or additively, the patient may suffer from any one or more of pulmonary arterial hypertension, chronic thromboembolic pulmonary hypertension, pulmonary hypertension, persistent pulmonary hypertension of the new born, portal hypertension, pulmonary hypertension—left ventricular systolic dysfunction, pulmonary hypertension—idiopathic interstitial pneumonias, diffuse cutaneous systemic sclerosis, cystic fibrosis, moyamoya syndrome, sickle cell disease, erectile dysfunction, heart failure with reduced ejection fraction, heart failure with preserved ejection fraction, type 2 diabetes mellitus, hypertension, acute decompensated chronic congestive heart failure, moderate calcific aortic valve stenosis, peripheral arterial disease, erectile dysfunction, fibrotic conditions such as liver fibrosis, NASH, complications relating to diabetes mellitus, such as diabetic nephropathy and diabetic cardiomyopathy, and COVID19-related respiratory distress and/or cardiovascular complications, preferably any one or more of ischemia, neonatal asphyxia, oxidative organ damage, oxidative tissue damage, oxidative cell damage, stroke, acute respiratory distress syndrome and asthma, preferably ischemia, oxidative organ damage, oxidative tissue damage, oxidative cell damage and stroke, more preferably ischemia and stroke.

It is part of the invention that the sGCs compound(s) comprised by any of the pharmaceutical compositions of the invention or comprised by any of the dose combinations of the invention are administered to the patient at lower dose than doses that are currently commonly administered to patients in need thereof, or which are tested in clinical trials. That is to say, the effective dose of the sGCs in the pharmaceutical compositions of the invention and in the dose combinations of the invention is lower than what is common until the present invention became apparent. For example, the effective dose for a single administration to a patient in need thereof, of riociguat is 5 mg/dose or lower, for example administered once, twice, thrice daily to the patient, preferably once daily, preferably less than 5 mg/dose, less than 4 mg/dose, less than 3 mg/dose, less than 2.5 mg/dose, less than 2.0 mg/dose, less than 1.5 mg/dose, less than 1.0 mg/dose, preferably less than 0.5 mg/dose. Further examples are nelociguat, for which an effective dose unit for administration one-thrice daily to a patient in need thereof, is 1.0 mg/dose or less such as less than 0.8 mg/dose, less than 0.5 mg/dose; vericiguat, for which an effective dose unit for administration one-thrice daily to a patient in need thereof, is 15 mg/dose or less such as less than 10 mg/dose, less than 5 mg/dose, less than 2.5 mg/dose, less than 1.25 mg/dose such as 0.2 mg/dose-1.0 mg/dose; olinciguat, for which an effective dose unit for administration one-thrice daily to a patient in need thereof, is 5 mg/dose or less such as less than 4 mg/dose, less than 3 mg/dose, such as 0.5 mg/dose-2.5 mg/dose; praliciguat, for which an effective dose unit for administration one-thrice daily to a patient in need thereof, is 50 mg/dose or less such as less than 40 mg/dose, less than 30 mg/dose, less than 20 mg/dose, less than 10 mg/dose, such as 1 mg/dose-6 mg/dose.

Such lower doses than commonly applied are now still effective and applicable for the treatment of patients in need thereof since the inventors established that the sGCs synergistically exerts its activity together with a further (low-dose) sGCs and/or in a combination therapy with at least one (low-dosed) sGCa. Typically, the patient in need of such treatment is a CVD patient such as a patient suffering from cGMP deficiency, stroke, hypoxia, heart infarct, oxidative damage, post-reperfusion oxidative damage, (abundant) presence of apo-sGC, (risk for) hypotension, NO-insensitive sGC. Typically, the single sGCs supplied to the patient at low dose or the combination of two or more sGCs, at least one and preferably both or all administered to the patient at low dose, are used in a method for the treatment of e.g. a CVD, wherein the patient typically suffers from the abundant presence of apo-sGC, for which patient typically previously treatment encompassing administering an sGCa compound would have been selected before the current invention became apparent. Preferred is the sGCs for use in a method for the treatment of a CVD accompanied by the presence of apo-sGC in the patient in need of treatment, wherein the sGCs therapy is combined with sGCa therapy.

It is part of the invention that the sGCa compound(s) comprised by any of the pharmaceutical compositions of the invention or comprised by any of the dose combinations of the invention are administered to the patient at lower dose than doses that are currently commonly administered to patients in need thereof, or which are tested in clinical trials. That is to say, the effective dose of the sGCa in the pharmaceutical compositions of the invention and in the dose combinations of the invention is lower than what is common until the present invention became apparent. For example, the effective dose for a single administration to a patient in need thereof, of ataciguat is 200 mg/dose or less, for example administered once, twice, thrice daily to the patient, preferably once daily, preferably less than 100 mg/dose, less than 50 mg/dose, less than 40 mg/dose, less than 30 mg/dose, less than 20 mg/dose, less than 15 mg/dose, less than 10 mg/dose, preferably less than 5 mg/dose. Other exemplifying sGCa compounds applicable at low dose for administering to the patients in need thereof are listed in for example Table 2.

Typically, according to the invention, an sGCs compound is administered to a patient in need thereof, suffering from a disorder accompanied with the presence of apo-sGC, wherein the effective dose of the sGCs is at most half of the dose commonly administered to a patient in need of improving sGC activity under NO-insensitive conditions. Typically, according to the invention, an sGCs compound in combination with an sGCa compound (either as a single pharmaceutical composition, or as a fixed dose combination such as an oral dose combination) is administered to a patient in need thereof, suffering from a disorder accompanied with the presence of apo-sGC, wherein the effective dose of the sGCs is at most half of the dose commonly administered to a patient in need of improving sGC activity under NO-insensitive conditions, and wherein the effective dose of the sGCa is at most half of the dose commonly administered to a patient in need of improving sGC activity when the presence of apo-sGC in the patient is apparent. Such lower than common practice-doses of sGCs and/or sGCa administered to a patient in need thereof, are now become possible due to the invention, since the inventors gained the insight that in patients suffering from a disorder or disease accompanied with presence of apo-sGC, the sGCs and the sGCa exhibit synergistic sGC stimulatory activity, and thus exhibit synergistically improved cGMP production in the patient.

It is one of the many benefits for the patient, now becoming available due to the present invention, that therapy with either a single sGCs, or more than one sGCs, or a single sGCs and an sGCa, etc., is efficacious and effective even when the sGCs and/or the sGCa are administered to the patient in need thereof at a lower dose than what was the standard practice before. Such lower doses of the sGCs and/or the sGCa lower the risk for occurrence of adverse effects and unacceptable or unwanted side effects due to the (once, twice of thrice daily) administration of the oral doses of the one or more (apo-)sGC stimulatory compounds. Synergy between two or more sGCs compounds and between sGCs compounds and sGCa compounds beneficially provides for an improvingly effective therapy for the patient with less burden due to side effects and with less risk for the occurrence of adverse events due to the lower (daily) doses of the activator(s) and/or stimulator(s) of cGMP production.

According to the invention, typical pharmaceutical compositions and typical dose combinations comprise any one or more of Riociguat, Vericiguat, BAY 60-4552, YC-1, A-350619, CF-1571, Olinciguat, Praliciguat either or not in combination with any one or more of Cinaciguat, HMR 1766, BI 703704, BI 684067. Pharmaceutical compositions of the invention and (oral) dose combinations of the invention for example comprise the following combinations of two sGCs: Riociguat, Vericiguat; Riociguat, BAY 60-4552; Riociguat, YC-1; Riociguat, A-350619; Riociguat, CF-1571; Riociguat, Olinciguat; Riociguat, Praliciguat; Vericiguat, BAY 60-4552; Vericiguat, YC-1; Vericiguat, A-350619; Vericiguat, CF-1571; Vericiguat, Olinciguat; Vericiguat, Praliciguat; BAY 60-4552, YC-1; BAY 60-4552, A-350619; BAY 60-4552, CF-1571; BAY 60-4552, Olinciguat; BAY 60-4552, Praliciguat; YC-1, A-350619; YC-1, CF-1571; YC-1, Olinciguat; YC-1, Praliciguat; A-350619, CF-1571; A-350619, Olinciguat; A-350619, Praliciguat; CF-1571, Olinciguat; CF-1571, Praliciguat; Olinciguat, Praliciguat, or of one sGCs and one sGCa, Riociguat, Cinaciguat; Riociguat, HMR 1766; Riociguat, BI 703704; Riociguat, BI 684067; Vericiguat, Cinaciguat; Vericiguat, HMR 1766; Vericiguat, BI 703704; Vericiguat, BI 684067; BAY 60-4552, Cinaciguat; BAY 60-4552, HMR 1766; BAY 60-4552, BI 703704; BAY 60-4552, BI 684067; YC-1, Cinaciguat; YC-1, HMR 1766; YC-1, BI 703704; YC-1, BI 684067; A-350619, Cinaciguat; A-350619, HMR 1766; A-350619, BI 703704; A-350619, BI 684067; CF-1571, Cinaciguat; CF-1571, HMR 1766; CF-1571, BI 703704; CF-1571, BI 684067; Olinciguat, Cinaciguat; Olinciguat, HMR 1766; Olinciguat, BI 703704; Olinciguat, BI 684067; Praliciguat, Cinaciguat; Praliciguat, HMR 1766; Praliciguat, BI 703704; Praliciguat, BI 684067.

Typically, according to the invention, a pharmaceutical composition or a dose combination is provided as tablets, pills, a powder, capsules, wherein preferably a single tablet, pill, packaged amount of powder, capsule, comprises a single dose unit of the active pharmaceutical ingredient, i.e. one or more sGCs and/or sGCa. Alternatively, a combination of two or more tablets, capsules, etc., provides a single dosage of the API(s), such as two, three, four or five capsules, tablets, etc. Typically and preferred, the pharmaceutical composition or the dose combination is to be administered once daily to a patient in need thereof in order to treat the patient with an effective daily dose of the one or more sGCs and one or more sGCa. Alternatively, the pharmaceutical composition or the dose combination is to be administered twice or thrice daily to a patient in need thereof.

An aspect of the invention relates to a therapeutic combination comprising:
a. a first unit dose comprising:
   i. a first sGCs;
   ii. optionally a second sGCs; and either
b. a second unit dose comprising:
   i. a first sGCa;
   ii. optionally a second sGCa; or
c. a third unit dose comprising:
   iii. a third sGCs; and
   iv. optionally a fourth sGCs.

An embodiment is the therapeutic combination of the invention, wherein the first, second, third and fourth sGCs is any of Riociguat, Vericiguat, BAY 60-4552, YC-1, A-350619, CF-1571, Olinciguat, Praliciguat, wherein the first and second sGCa is any of Cinaciguat, HMR 1766, BI 703704, BI 684067, if the second unit dose is present in the therapeutic combination. Preferred is the therapeutic combination , wherein the first, second, third and fourth sGCs is any of Riociguat, Vericiguat, BAY 60-4552, YC-1, A-350619, CF-1571, Olinciguat, Praliciguat, wherein the first and second sGCa is any of Cinaciguat, HMR 1766, BI 703704, BI 684067 and Bay 12-11163, preferably the sGCs is Riociguat or Vericiguat and the sGCa is Bay 12-11163.

An embodiment is the therapeutic combination of the invention, wherein the amount sGCs per unit dose in the first and/or third unit dose is for riociguat 5 mg or lower, preferably less than 4 mg, less than 3 mg, less than 2.5 mg, less than 2.0 mg, less than 1.5 mg, less than 1.0 mg, preferably less than 0.5 mg; for nelociguat 1.0 mg or less such as less than 0.8 mg, less than 0.5 mg; for vericiguat 15 mg or less such as less than 10 mg, less than 5 mg, less than 2.5 mg, less than 1.25 mg such as 0.2 mg-1.0 mg; for olinciguat 5 mg or less such as less than 4 mg, less than 3 mg, such as 0.5 mg-2.5 mg; of praliciguat 50 mg or less such as less than 40 mg, less than 30 mg, less than 20 mg, less than 10 mg, such as 1 mg-6 mg; and wherein the amount sGCa per unit dose in the second unit dose is for ataciguat 200 mg/dose or less, preferably less than 100 mg/dose, less than 50 mg/dose, less than 40 mg/dose, less than 30 mg/dose, less than 20 mg/dose, less than 15 mg/dose, less than 10 mg/dose, preferably less than 5 mg/dose.

An embodiment is the therapeutic combination of the invention, comprising a first unit dose and a second unit dose, the first and second unit dose comprising respectively any combination of Riociguat, Cinaciguat; Riociguat, HMR 1766; Riociguat, BI 703704; Riociguat, BI 684067; Vericiguat, Cinaciguat; Vericiguat, HMR 1766; Vericiguat, BI 703704; Vericiguat, BI 684067; BAY 60-4552, Cinaciguat; BAY 60-4552, HMR 1766; BAY 60-4552, BI 703704; BAY 60-4552, BI 684067; YC-1, Cinaciguat; YC-1, HMR 1766; YC-1, BI 703704; YC-1, BI 684067; A-350619, Cinaciguat; A-350619, HMR 1766; A-350619, BI 703704; A-350619, BI 684067; CF-1571, Cinaciguat; CF-1571, HMR 1766; CF-1571, BI 703704; CF-1571, BI 684067; Olinciguat, Cinaciguat; Olinciguat, HMR 1766; Olinciguat, BI 703704; Olinciguat, BI 684067; Praliciguat, Cinaciguat; Praliciguat, HMR 1766; Praliciguat, BI 703704; or Praliciguat, BI 684067; or comprising a first unit dose and a third unit dose, the first and third unit dose comprising respectively any combination of Riociguat, Vericiguat; Riociguat, BAY 60-4552; Riociguat, YC-1; Riociguat, A-350619; Riociguat, CF-1571; Riociguat, Olinciguat; Riociguat, Praliciguat; Vericiguat, BAY 60-4552; Vericiguat, YC-1; Vericiguat, A-350619; Vericiguat, CF-1571; Vericiguat, Olinciguat; Vericiguat, Praliciguat; BAY 60-4552, YC-1; BAY 60-4552, A-350619; BAY 60-4552, CF-1571; BAY 60-4552, Olinciguat; BAY 60-4552, Praliciguat; YC-1, A-350619; YC-1, CF-1571; YC-1, Olinciguat; YC-1, Praliciguat; A-350619, CF-1571; A-350619, Olinciguat; A-350619, Praliciguat; CF-1571, Olinciguat; CF-1571, Praliciguat; or Olinciguat, Praliciguat. Preferred is the therapeutic combination of the invention, comprising a first unit dose and a second unit dose, the first and second unit dose comprising respectively any combination of Riociguat, Cinaciguat; Riociguat, HMR 1766; Riociguat, BI 703704; Riociguat, BI 684067; Riociguat, Bay 12-11163; Vericiguat, Cinaciguat; Vericiguat, HMR 1766; Vericiguat, BI 703704; Vericiguat, BI 684067; Vericiguat, Bay 12-11163; BAY 60-4552, Cinaciguat; BAY 60-4552, HMR 1766; BAY 60-4552, BI 703704; BAY 60-4552, BI 684067; BAY 60-4552, Bay 12-11163; YC-1, Cinaciguat; YC-1, HMR 1766; YC-1, BI 703704; YC-1, BI 684067; YC-1, Bay 12-11163; A-350619, Cinaciguat; A-350619, HMR 1766; A-350619, BI 703704; A-350619, BI 684067; CF-1571, Cinaciguat; CF-1571, HMR 1766; CF-1571, BI 703704; CF-1571, BI 684067; Olinciguat, Cinaciguat; Olinciguat, HMR 1766; Olinciguat, BI 703704; Olinciguat, BI 684067; Praliciguat, Cinaciguat; Praliciguat, HMR 1766; Praliciguat, BI 703704; or Praliciguat, BI 684067; or comprising a first unit dose and a third unit dose, the first and third unit dose comprising respectively any combination of Riociguat, Vericiguat; Riociguat, BAY 60-4552; Riociguat, YC-1; Riociguat, A-350619; Riociguat, CF-1571; Riociguat, Olinciguat; Riociguat, Praliciguat; Vericiguat, BAY 60-4552; Vericiguat, YC-1; Vericiguat, A-350619; Vericiguat, CF-1571; Vericiguat, Olinciguat; Vericiguat, Praliciguat; BAY 60-4552, YC-1; BAY 60-4552, A-350619; BAY 60-4552, CF-1571; BAY 60-4552, Olinciguat; BAY 60-4552, Praliciguat; YC-1, A-350619; YC-1, CF-1571; YC-1, Olinciguat; YC-1, Praliciguat; A-350619, CF-1571; A-350619, Olinciguat; A-350619, Praliciguat; CF-1571, Olinciguat; CF-1571, Praliciguat; or Olinciguat, Praliciguat.

An aspect of the invention relates to the therapeutic combination of the invention, for use as a medicament.

An aspect of the invention relates to the therapeutic combination of the invention for use in a method for the treatment of cyclic 3′,5′-guanosine monophosphate (cGMP) deficiency in a patient, preferably a human patient.

An embodiment is the therapeutic combination for use according to the invention, wherein the use is in a method for the treatment of a cardiovascular disease, or wherein the patient deficient in cGMP suffers from a cardiovascular disease.

An embodiment is the therapeutic combination for use according to the invention, wherein the patient to whom the therapeutic combination is administered, suffers from any one or more of pulmonary arterial hypertension, chronic thromboembolic pulmonary hypertension, pulmonary hypertension, persistent pulmonary hypertension of the new born, portal hypertension, pulmonary hypertension—left ventricular systolic dysfunction, pulmonary hypertension—idiopathic interstitial pneumonias, diffuse cutaneous systemic sclerosis, cystic fibrosis, moyamoya syndrome, sickle cell disease, erectile dysfunction, heart failure with reduced ejection fraction, heart failure with preserved ejection fraction, type 2 diabetes mellitus, hypertension, acute decompensated chronic congestive heart failure, moderate calcific aortic valve stenosis, peripheral arterial disease, erectile dysfunction, fibrotic conditions such as liver fibrosis, NASH, complications relating to diabetes mellitus, such as diabetic nephropathy and diabetic cardiomyopathy, and COVID19-related respiratory distress and/or cardiovascular complications, ischemia, neonatal asphyxia, oxidative organ damage, oxidative tissue damage, oxidative cell damage, stroke, acute respiratory distress syndrome and asthma, preferably ischemia, oxidative organ damage, oxidative tissue damage, oxidative cell damage and stroke, more preferably ischemia and stroke.

An embodiment is the therapeutic combination for use according to the invention, wherein the therapeutic combination wherein the first, second, third unit dose is administered to the patient as a single solid dosage daily, or as two-four solid dosages daily, such as thrice daily, and wherein the therapeutic combination is for oral administration.

An embodiment is the therapeutic combination for use according to the invention, wherein the patient in need thereof is administered an effective dose of the therapeutic combination of the invention.

An embodiment is the therapeutic combination for use according to the invention, wherein the patient suffers from a disease or disorder accompanied by the presence of apo-sGC. Preferred is the therapeutic combination for use according to the invention, wherein the patient suffers from a disease or disorder accompanied by the presence of apo-sGC, such as any one or more of ischemia, neonatal asphyxia, oxidative organ damage, oxidative tissue damage, oxidative cell damage, stroke, acute respiratory distress syndrome and asthma, preferably ischemia, neonatal asphyxia, oxidative organ damage, oxidative tissue damage, oxidative cell damage and stroke, more preferably ischemia and stroke, and optionally wherein the patient suffers from a disease or disorder accompanied by the presence of apo-sGC and the absence of sGC.

An embodiment is the therapeutic combination for use according to the invention, wherein the treatment comprises stimulation of cGMP formation in the patient in need of said treatment.

An embodiment is the therapeutic combination for use according to the invention, wherein the patient suffers from NO insufficiency and/or from oxidative damage.

An embodiment is the therapeutic combination for use according to the invention, wherein the patient suffers from a medical condition relating to sGC dysfunction and/or relating to cGMP deficiency.

An embodiment is the therapeutic combination for use according to the invention, wherein the sGCs augment(s) stimulation of heme containing sGC and augment(s) stimulation of sGC by NO and/or stimulate(s) apo-sGC.

An embodiment is the therapeutic combination for use according to the invention, wherein the one or more sGCs and the one or more sGCa augment sGC and/or apo-sGC synergistically.

An aspect of the invention relates to a therapeutic combination comprising:
a. a first unit dose comprising:
   i. a first sGCs;
   ii. optionally a second sGCs; and
b. a second unit dose comprising:
   i. a first NO donor compound;
   ii. optionally a second NO donor compound; and optionally comprising
c. a third unit dose comprising:
   iii. a first sGCa; and
   iv. optionally a second sGCa.

An embodiment is the therapeutic combination of the invention, wherein the first and if present the second sGCs is any of Riociguat, Vericiguat, BAY 60-4552, YC-1, A-350619, CF-1571, Olinciguat, Praliciguat, and when present, wherein the first and second sGCa is any of Cinaciguat, HMR 1766, BI 703704, BI 684067. Preferred is the therapeutic combination, wherein the first and if present the second sGCs is any of Riociguat, Vericiguat, BAY 60-4552, YC-1, A-350619, CF-1571, Olinciguat, Praliciguat, preferably Riociguat and/or Vericiguat, and when present, wherein the first and second sGCa is any of Cinaciguat, HMR 1766, BI 703704, BI 684067 and Bay 12-11163.

An embodiment is the therapeutic combination of the invention, wherein the amount sGCs per unit dose in the first unit dose is for riociguat 5 mg or lower, preferably less than 4 mg, less than 3 mg, less than 2.5 mg, less than 2.0 mg, less than 1.5 mg, less than 1.0 mg, preferably less than 0.5 mg; for nelociguat 1.0 mg or less such as less than 0.8 mg, less than 0.5 mg; for vericiguat 15 mg or less such as less than 10 mg, less than 5 mg, less than 2.5 mg, less than 1.25 mg such as 0.2 mg-1.0 mg; for olinciguat 5 mg or less such as less than 4 mg, less than 3 mg, such as 0.5 mg-2.5 mg; of praliciguat 50 mg or less such as less than 40 mg, less than 30 mg, less than 20 mg, less than 10 mg, such as 1 mg-6 mg; and if present wherein the amount sGCa per unit dose in the third unit dose is for ataciguat 200 mg/dose or less, preferably less than 100 mg/dose, less than 50 mg/dose, less than 40 mg/dose, less than 30 mg/dose, less than 20 mg/dose, less than 15 mg/dose, less than 10 mg/dose, preferably less than 5 mg/dose.

An embodiment is the therapeutic combination of the invention, wherein the first NO donor compound and when present the second NO donor compound is/are selected from an organic nitrate, an organic nitrite, an S-nitrosothiol, a prusside, an NONOate, a sydnonimine, an oxatriazole, a furoxan, a Ruthenium nitrosyl, a photochemical donor via one/two-photon excitation, a diazeniumdiolated carbamate, nitroglycerin, molsidomine, isosorbide dinitrate (ISDN), sodium nitroprusside or an alternative pharmaceutically acceptable nitroprusside salt, and any pharmaceutically acceptable derivative thereof and/or any pharmaceutically acceptable salt thereof and/or any pharmaceutically acceptable prodrug thereof.

An aspect of the invention relates to the therapeutic combination of the invention, for use as a medicament.

An embodiment is the therapeutic combination of the invention for use of the invention, wherein the use is in a method for the treatment of cyclic 3′,5′-guanosine monophosphate (cGMP) deficiency in a patient, preferably a human patient.

An embodiment is the therapeutic combination for use according to the invention, wherein the use is in a method for the treatment of a cardiovascular disease, or wherein the patient deficient in cGMP suffers from a cardiovascular disease.

An embodiment is the therapeutic combination for use according to the invention, wherein the patient to whom the therapeutic combination is administered, suffers from any one or more of pulmonary arterial hypertension, chronic thromboembolic pulmonary hypertension, pulmonary hypertension, persistent pulmonary hypertension of the new born, portal hypertension, pulmonary hypertension—left ventricular systolic dysfunction, pulmonary hypertension—idiopathic interstitial pneumonias, diffuse cutaneous systemic sclerosis, cystic fibrosis, moyamoya syndrome, sickle cell disease, erectile dysfunction, heart failure with reduced ejection fraction, heart failure with preserved ejection fraction, type 2 diabetes mellitus, hypertension, acute decompensated chronic congestive heart failure, moderate calcific aortic valve stenosis, peripheral arterial disease, erectile dysfunction, fibrotic conditions such as liver fibrosis, NASH, complications relating to diabetes mellitus, such as diabetic nephropathy and diabetic cardiomyopathy, and COVID19-related respiratory distress and/or cardiovascular complications, ischemia, neonatal asphyxia, oxidative organ damage, oxidative tissue damage, oxidative cell damage, stroke, acute respiratory distress syndrome and asthma, preferably ischemia, neonatal asphyxia, oxidative organ damage, oxidative tissue damage, oxidative cell damage and stroke, more preferably ischemia and stroke. Preferred is the therapeutic combination for use, wherein the patient to whom the therapeutic combination is administered, suffers from any one or more of pulmonary arterial hypertension, chronic thromboembolic pulmonary hypertension, persistent pulmonary hypertension of the new born, portal hypertension, pulmonary hypertension, pulmonary hypertension—left ventricular systolic dysfunction, pulmonary hypertension—idiopathic interstitial pneumonias, diffuse cutaneous systemic sclerosis, cystic fibrosis, sickle cell disease, erectile dysfunction, heart failure with reduced ejection fraction, heart failure with preserved ejection fraction, type 2 diabetes mellitus, hypertension, acute decompensated chronic congestive heart failure, moderate calcific aortic valve stenosis, peripheral arterial disease, and/or suffers from any one or more of oxidative damage, ischemia, neonatal asphyxia, oxidative organ damage, oxidative tissue damage, oxidative cell damage, stroke, acute respiratory distress syndrome and asthma, preferably from any one or more of nitric oxide (NO) insufficiency and/or ischemia, neonatal asphyxia, oxidative organ damage, oxidative tissue damage, oxidative cell damage and stroke, more preferably from nitric oxide (NO) insufficiency and/or ischemia and/or stroke.

An embodiment is the therapeutic combination for use according to the invention, wherein the therapeutic combination wherein the first, second, third unit dose is administered to the patient as a single solid dosage daily, or as two-four solid dosages daily, such as thrice daily, and wherein the therapeutic combination is for oral administration.

An embodiment is the therapeutic combination for use according to the invention, wherein the patient in need thereof is administered an effective dose of said therapeutic combination.

An embodiment is the therapeutic combination for use according to the invention, wherein the patient suffers from a disease or disorder accompanied by the presence of apo-sGC. Preferred is the therapeutic combination for use according to the invention, wherein the patient suffers from a disease or disorder accompanied by the presence of apo-sGC, such as any one or more of ischemia, neonatal asphyxia, oxidative organ damage, oxidative tissue damage, oxidative cell damage, stroke, acute respiratory distress syndrome and asthma, preferably ischemia, neonatal asphyxia, oxidative organ damage, oxidative tissue damage, oxidative cell damage and stroke, more preferably ischemia and stroke, and optionally wherein the patient suffers from a disease or disorder accompanied by the presence of apo-sGC and the absence of sGC.

An embodiment is the therapeutic combination for use according to the invention, wherein the treatment comprises stimulation of cGMP formation in the patient in need of said treatment.

An embodiment is the therapeutic combination for use according to the invention, wherein the patient suffers from NO insufficiency and/or from oxidative damage. Preferred is the therapeutic combination for use, wherein the patient suffers from NO insufficiency and/or from oxidative damage, ischemia, neonatal asphyxia, oxidative organ damage, oxidative tissue damage, oxidative cell damage, stroke, acute respiratory distress syndrome and asthma, preferably from any one or more of nitric oxide (NO) insufficiency and/or ischemia, neonatal asphyxia, oxidative organ damage, oxidative tissue damage, oxidative cell damage and stroke, more preferably from nitric oxide (NO) insufficiency and/or ischemia and/or stroke.

An embodiment is the therapeutic combination for use according to the invention, wherein the patient suffers from a medical condition relating to sGC dysfunction and/or relating to cGMP deficiency.

An embodiment is the therapeutic combination for use according to the invention, wherein the sGCs augment(s) stimulation of heme containing sGC and augment(s) stimulation of sGC by NO and/or stimulate(s) apo-sGC.

An embodiment is the therapeutic combination for use according to the invention, wherein the one or more sGCs and the one or more sGCa augment sGC and/or apo-sGC synergistically.

As exemplified in the Example section here below, and in particular in FIG. 12, the inventors surprisingly found that apo-sGC activation and stimulation, resulting in cGMP production by cells, synergistically increased when those cells had apo-sGC and were stimulated with a combination of an NO donor compound (here, DETA-NONOate) and an sGC stimulator (here, Bay41-2272). The cells hardly produced any cGMP upon stimulation of the apo-sGC with the NO donor compound only. Stimulation of the apo-sGC with the sGC stimulator resulted in cGMP production by the cells. Combining the sGC stimulator with the NO donor compound resulted in a more than double amount of cGMP, demonstrating the synergistic manner in which the sGC stimulator compound and the NO donor compound are able to activate the apo-sGC. In particular, reference is made to FIG. 12A and B.

### Embodiments

Stroke is the leading cause of disability and represents one of the largest unmet medical needs as only one drug is available for treatment. This drug is limited to the acute phase of stroke and dissolves clots that reduce blood flow to the brain. It is, however, only marginally effective, bears a high risk of fatal bleeding and has over 30 contraindications, which is why most stroke patients are not treated with it. There is a strong need for a stroke drug that is broadly applicable, and/or has few or no contraindications, and/or bears no bleeding risk, and/or reduces brain damage and/or improves brain function, preferably a stroke drug that fulfils all of these aspects beneficial to the patient to be treated. The inventors found a therapeutic approach that, surprisingly, fulfils all of the above criteria and that is also innovative from a commercial perspective, and that is rapidly applicable in the clinic. The current invention relates to the field of repurposing of drugs that are already registered but for a different indication than stroke. By combining two or three selected optimal compounds, the risk of the frequent failure of single compounds in drug development is reduced. Without wishing to be bound by any theory, all two or three chosen compounds according to the invention are strongly neuroprotective on their own; all compounds target the same disease mechanism, yet at different positions and thereby potentiate each other according to embodiments of the invention, which increases the chance of therapeutic success such as therapeutic success in clinical studies. The inventors were, to their surprise, also able to lower the dose of each compound, which lowers the risk of possible side effects. In preparation of a clinical trial, which for regulatory requirements has to have safety as primary outcome, the inventors extended the conducted small-animal validation data by conducting a successful large animal safety study with two of the compounds that were suitable for administration in sheep. Moreover, through plasma biomarkers measured in biobank samples from stroke patients the inventors narrowed down the ideal time-window up to which the drug combination is highly likely to be effective, according to the invention. Reference is made to the Examples and to the claims.

### Examples and Exemplary Embodiments

### Experimental Designs

We tested two representative compounds of each drug class, the sGC activator BAY58-2667 and the sGC stimulator BAY 41-2272, individually in different models and in a range of concentrations. We studied whether a combination of BAY 58-2667 and BAY41-2272 could lead to supra-additive effects. In rat mesenteric arteries, BAY 41-2272 augmented both DEA/NO (NO donor) and Bay 58-2667 relaxation in the nano-molar range. Using another model i.e. lung homogenates of sGCβ31H105F knock-in (apo-sGC) mice, the potentiation of BAY 58-2667 by BAY 41-2272 could be confirmed, again with maximal efficiency in the nano-molar range. In summary, our data suggest a combination of sGC activator and stimulator at concentrations that lead to synergy and maximal efficiency, thereby allowing to use both drugs at lower concentrations.

### Experimental Methods

### Chemicals

Polyclonal antibodies specific for sGCβ31 and sGCα1 have been described elsewhere (Ibarra et al., Brain Res. 2001; 907(1-2):54-60. doi: 10.1016/S0006-8993(01)02588-4. IBMX and GTP (Enzo LifeSciences, Lörrach, Germany); BAY 58-2667 (4-[((4-carboxybutyl){2-[(4-phenethylbenzyl)oxy] phenethyl}amino) methyl[benzoic]acid) and BAY 41-2272 (5-cyclopropyl-2-[1-(2-fluoro-benzyl)-1H-pyrazolo[3,4-b]pyridin-3-yl]-pyrimidin-4-ylamine) were synthesized as described in international patent applications WO/2001/019776 and WO/2001/083490. All other chemicals were of the highest purity grade available and obtained from either Sigma Chemicals (Deisenhofen, Germany) or Merck AG (Darmstadt, Germany). BAY 58-2667 and BAY 41-2272 were dissolved in DMSO. BAY 60-2770 has been obtained by Bayer.

### Animals

Adult male Sprague Dawley (SD) rats (age, 10-12 weeks) were obtained from Animal Resources Centre (Western Australia). Animals were housed on a 12 hour day/night cycle at a room temperature of 20±2° C., in the Department of Pharmacology Animal House, Monash University. The animals were fed standard rodent chow and water was available ad libitum. Homozygous male sGC(1^{H105F} knock-in mice (“apo-sGC mice”) were kindly provided by Tufts Medical Center, Molecular Cardiology Research Center, Boston, Mass., USA (Toonen et al., Nat Commun. 2015; 6:8482. doi: 10.1038/ncomms9482).

### Tissue Collection

The animals were killed humanely via CO2 inhalation (95% CO₂, 5% O₂) followed by cervical dislocation and exsanguination. The required tissues were dissected and placed in ice-cold oxygenated Krebs' solution (composition in mM: NaCl 119, KCl 4.7, MgSO₄ 1.17, NaHCO₃ 25, KH₂PO₄ 1.18, CaCl₂ 2.5, glucose 5.5, EDTA 0.026, pH 7.4). The collected arteries were carefully freed of fat and connective tissue and cut into 2 mm rings.

### Determination of sGC Activity

To measure sGC activity, cells were stimulated with 250 μM DEA/NO or 10 μM BAY58-2667 for 3 min at 37° C. Thereafter, cells were immediately lysed in 80% ethanol. Cells were scraped and, after evaporation of ethanol, re-suspended in assay buffer and sonicated. Measurement of sGC activity in crude homogenates of mouse tissue was performed as previously described (Nedvetsky et al., Brain Res. 2002; 950(1-2):148-154. doi: 10.1016/50006-8993(02)03015-9).[9] Briefly, all samples were measured as the formation of cGMP at 37° C. during 10 min in a total incubation volume of 100 ml containing 50 mM triethanolamine-HCl (pH 7.4), 3 mM MgCl₂, 3 mM glutathione (Carl Roth, Karlsruhe, Germany), 1 mM IBMX, 100 mM zaprinast, 5 mM creatine phosphate, 0.25 mg/ml creatine kinase and 1 mM or 0.5 mM GTP. After adding sGC activator in the absence or presence of the sGC stimulator the reaction was started by application of GTP solution. Following incubation of each sample for 10 min the reaction was stopped by boiling for 10 min at 95° C. Thereafter the amount of cGMP was subsequently determined by a commercial enzyme immunoassay kit (Enzo Life Sciences, Lorrach, Germany or Biotrend, Cologne, Germany). In some experiments pre-incubation of freshly homogenized tissue with ODQ at 37° C. for 20 min prior measuring sGC activation was required.

### Myograph Studies

Small rat arteries (mesenteric arteries corresponding to a third order branch of the superior mesenteric artery) were dissected and cleared of fat and cut into 2 mm segments. The segments were then mounted in a small vessel myograph for the measurement of changes in isometric tension. Two 40 μm stainless steel wires were inserted through the lumen of the segment, one attached to an isometric force transducer and the other to a support driven by a micrometer. The vessels were maintained in Krebs' solution at 37° C. in 7 ml myograph chambers and were continuously bubbled with carbogen (95% O₂, 5% CO₂). Arteries were allowed to equilibrate for a 30 min period under zero force after which their internal diameter was normalised to an equivalent transmural pressure of 100 mmHg. Changes in isometric tension were reordered using a Myograph Interface Model 610 M version 2.2 (AD Instruments, Pty Ltd) and a chart recorder (Yokogawa, Japan), or using a MacLab® (MacLab® data-acquisition system, AD Instruments Pty Ltd.), interfaced with a Macintosh Computer. Following the 30 min equilibration period at optimal resting tension, the vessels were contracted maximally using a potassium depolarising KPSS solution (composition in mM: KCl 123, MgSO₄ 1.17, KH₂PO₄ 2.37, CaCl₂ 2.5, glucose, 5.5 and EDTA 0.026). After the maximum contraction reached a plateau, the vessels were washed thoroughly using Krebs' solution and the tension allowed to return to the baseline. The vessels were then pre-contracted to ≈50% maximum contraction using titrated concentration of the thromboxane A2 mimetic U46619 (0.1-100 nM) and a titrated concentration of the al agonist cirazoline (1-100 nM). Subsequently, cumulative (0.5 log unit) concentration response curve to BAY 58-2667 (1 pM-0.1 μM) were constructed in the presence and absence of 100nM BAY 41-2272 (100 nM). All treatments used in our study including the pre-incubation with 10 μM ODQ for 30 min were added before the 50% pre-contraction with cirazoline and U46619 and only one concentration response curve to any vasodilator was constructed in each segment of the blood vessel. At the completion of each concentration response curve, maximal relaxation was achieved by adding nifedipine 10 μM.

### Generation of apo-sGC

Recombinant baculoviruses containing the cDNAs for the sGCa1 subunit with a His tag and mutant sGCβ31H105F with a His tag of human sGC were provided by E. Martin (Houston, Tex., USA). Sf9 cells (Thermo Fisher, Cat. no. 12659-017) were cultured in Sf-900 III serum-free medium supplemented with 2.5ml/l of 5000U/5000 μg penicillin-streptomycin. Spinner cultures were grown at 27° C. at 140 rpm shaking and diluted to 2×10⁶ cells/ml for infection. 50 ml Cell solution were infected with the respective recombinant baculovirus stock with a MOI of 0.1 for both baculoviruses. After 72 h cells were harvested and collected by centrifugation (4000×g for 10 min at 4° C.) and then stored at −80° C. The cell pellet was resuspended in homogenization buffer containing 50 mM triethanolamine/HCl, pH 7.5, 0.5 mM EDTA, 7 mM GSH, 0.2 mM PMSF, 1 μM pepstatinA, 1 μM leupeptin. The cells were lysed by sonication then centrifuged for 5 min at 13,000×g at 4° C. Supernatant was used for enzyme activity assay. Protein concentrations were determined by the RC DC protein assay kit (Bio-Rad, Cat. no. 5000122).

### Assay of cGMP Accumulation in Intact Sf9 Cells

Sf9 cells were co-infected with baculoviruses expressing human sGCa1 and mutant sGCβ1H105F. Forty-eight hours post-infection, the cells were treated with vehicle, NO donor, sGC stimulator, sGC activator or combination in 2 ml final volume of 10⁶ cells per ml then the cells were incubated for 10 min at 27° C. The reaction was terminated by the addition of 0.1M of HCl, and cGMP was extracted on ice for 20 min. Cells were then sonicated to ensure complete lysis. The extract was then centrifuged and used for cGMP determination by an ELISA (Enzo Life Sciences).

### Human Brain Microvascular Endothelial Cell (HBMEC) Cultures Subjected to Hypoxia

HBMEC (Cell systems, USA) between passage 3 and 9 were cultured to approximately 95% confluence using specialized cell medium (EGM-2 MV BulletKit, Lonza, The Netherlands) enriched with 5% fetal bovine serum before starting the hypoxia period. For hypoxia studies, HBMECs were seeded at specific density (6×10⁴ cells/ml) in 12 wells-plate and incubated during 24 h at 37° C. Then, cell medium was replaced with non-FBS containing medium following by 6 h of hypoxia (94.8% N₂, 0.2% O₂ and 5% CO₂) at 37° C. using hypoxia workstations (Ruskin Invivo2 400 station, The Netherlands). The hypoxia period was followed by 24 h of reperfusion in the presence or absence of 1 μM BAY 41-2272. Control cells were exposed to normoxia (75% N₂, 20% O₂ and 5% CO₂) and enriched medium during the hypoxia period.

### Assessment of Cell Viability in HBMEC

After 24 h of re-oxygenation period, cell viability was assessed using the colorimetric MTT assay. MTT solution (5 mg/ml) was added to each well (100 μl/ml) and incubated for 2 h at 37° C. The formazan salt formed was solubilized by adding 350 μl/well DMSO. The optical density was measured spectrophotometrically at 540 nm using a micro plate reader. Absorbance values obtained in control cells were set to 100% viability.

### In vivo MCAO Ischemia Model

C57B16/J mice were anesthetized with isoflurane (0.6% in oxygen). The animal was placed on a heating-pad, and rectal temperature was maintained at 37.0° C. Transient cerebral ischemia was induced using an intraluminal filament technique. Using a surgical microscope (Tecnoscopio OPMI pico, Carl Zeiss, Meditec Iberia SA, Spain), a midline neck incision was made and the right common and external carotid arteries were isolated and permanently ligated. A microvascular temporarily ligature was placed on the internal carotid artery to temporarily stop the blood flow. A silicon rubber-coated monofilament (6023910PK10, Doccol, USA) was inserted through a small incision into the common carotid artery and advanced into the internal carotid artery until a resistance is felt. The tip of the monofilament is then precisely located at the origin of the right middle cerebral artery. Animals were maintained under anaesthesia during 1 h occlusion period followed by the reperfusion period just started when the monofilament is removed. After the surgery, wounds were carefully sutured and animals could recover from surgery in a temperature-controlled cupboard. Riociguat was dissolved in saline (0.1 mg/kg) and injected i.p. 1 h after reperfusion.

### Determination of Infarct Size

After sacrificing the mice, brains were quickly removed and cut in four 2-mm thick coronal sections using a mouse brain slice matrix (Harvard Apparatus, USA). Brain slices were stained for 15 min at room temperature with 2% 2,3,5-triphenyltetrazolium chloride (TTC; Sigma-Aldrich, The Netherlands) in PBS to visualize the infarctions. Indirect infarct volumes were calculated by volumetry (ImageJ software, National Institutes of Health, USA) according to the following equation: V_{indirect} (mm³)=V_{infarct}×(1−(Vᵢₕ−V_{ch})/V_{ch}), where the term (Vᵢₕ−V_{ch}) represents the volume difference between the ischemic hemisphere and the control hemisphere and (Vᵢₕ−V_{ch})/V_{ch} expresses this difference as a percentage of the control hemisphere.

### Statistical Analysis

Results are expressed as mean±s.e.m, with n representing the number of samples used from separate subjects. Statistical significance was accepted at the P<0.05 level. Relaxation responses to BAY58-2667 were expressed as a percentage reversal of the level of pre-contraction to U46619, with the response to nifedipine 10 μM defined as 100% relaxation. The individual response curves obtained for each vasodilator were fitted using non-linear regressions (Graphpad Prism®, version 5). In-vitro experiments have been performed with n=3 and n=1. Maximal relaxation values (%) at specific concentrations of the compounds were compared. For multiple comparisons, student's t-test or one-way analysis of variance (ANOVA) was followed by Bonferroni's test.
The results of our experiments are represented by the following non-limiting examples.

### Example 1: Synergistic Activation of apo-sGC by sGC Activators and sGC Stimulators in Apo-sGC Mice

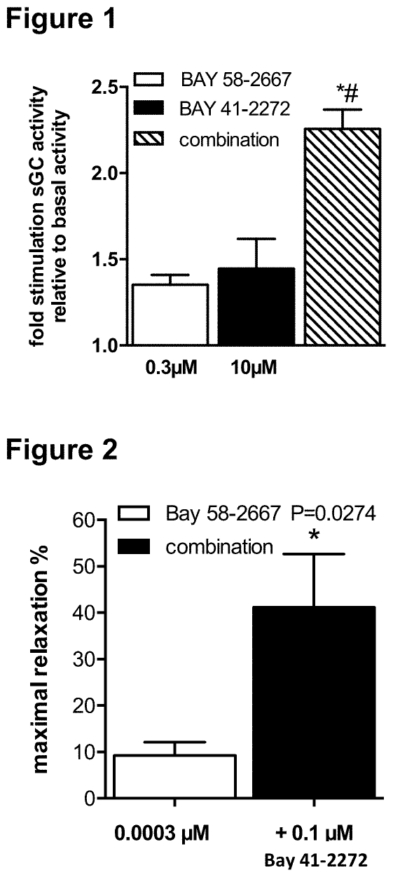

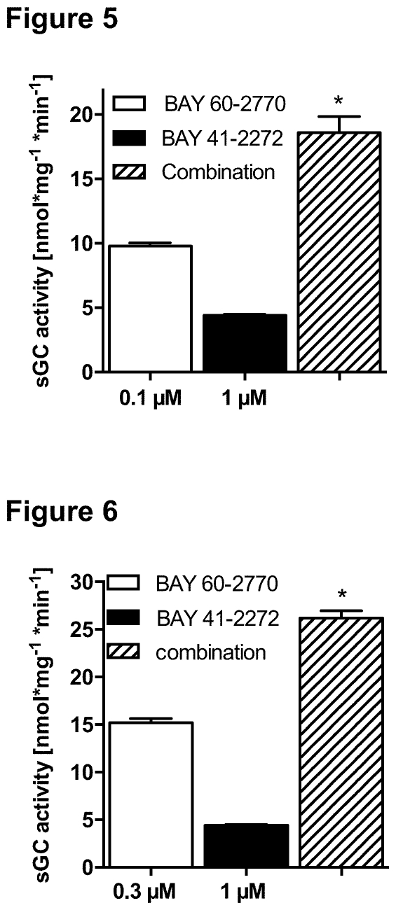

Oxidising Fe(II)sGC to its ferric form Fe(III)sGC can result in a rapid loss of its heme moiety, generating apo-sGC that is no longer NO responsive. This sGC redox state can be targeted by activators that bind specifically to the NO-insensitive, heme-free apo-sGC, thereby reactivating the oxidised and NO-insensitive enzyme and preventing its degradation via supra-physiological stabilization as seen for BAY 58-26673. On the other hand, sGC stimulators can bind NO-independently to yet unknown binding sites of sGC, leading to cGMP generation. To study whether a synergy of both principles can be achieved, we measured sGC activity in lung homogenates of apo-sGC mice, a strain that expresses mutant sGC without the NO binding site but is fully responsive to the sGC activator BAY58-2667 and can also be activated by the sGC stimulator BAY 41-2272. As shown in FIG. 1, the effect of 0.3 μM BAY 58-2667 could be potentiated in the presence of 10 μM BAY 41-2272.
A similar synergistic effect was observed when the sGC activator BAY 60-2770 (0.1 μM or 0.3 μM) was combined with 1 μM BAY 41-2772 (FIGS. 5 and 6).

### Example 2: Synergistic Effects on Blood Vessel Relaxation by BAY 58-2667 and BAY 41-2272 at Nanomolar Concentrations

In a subsequent set of experiments, we tested whether the relaxation effect of the sGC activator BAY 58-2667 on isolated rat mesenteric arteries can be potentiated by the sGC stimulator BAY 41-2272. For these ex vivo bioassays much lower concentrations were used than for the in-vitro assay.

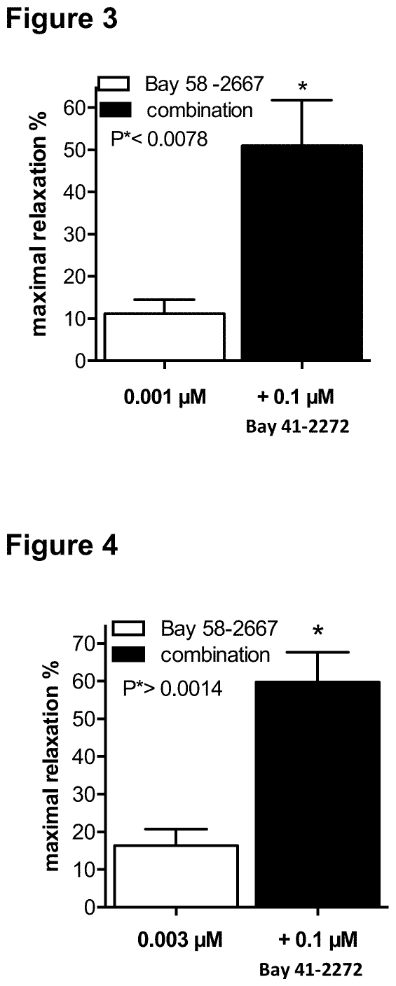

Super-additive effects could be observed when BAY 58-2667 (0.0003 μM, 0.001 μM, or 0.003 μM) was combined with 0.1 μM BAY 41-2272 (FIGS. 2-4).

### Example 3

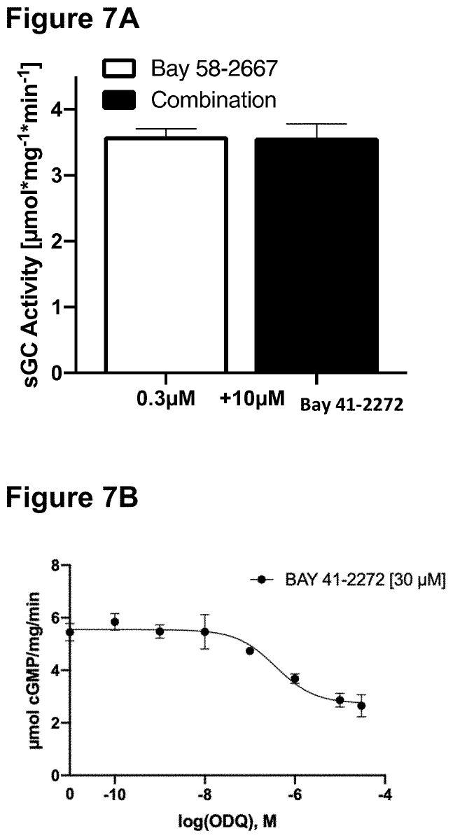

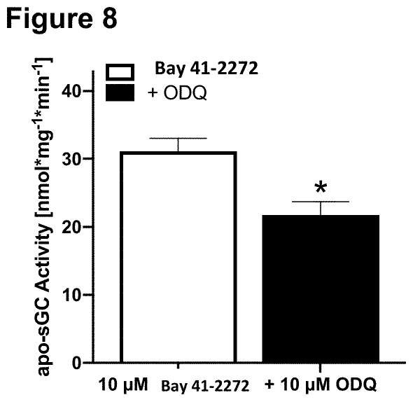

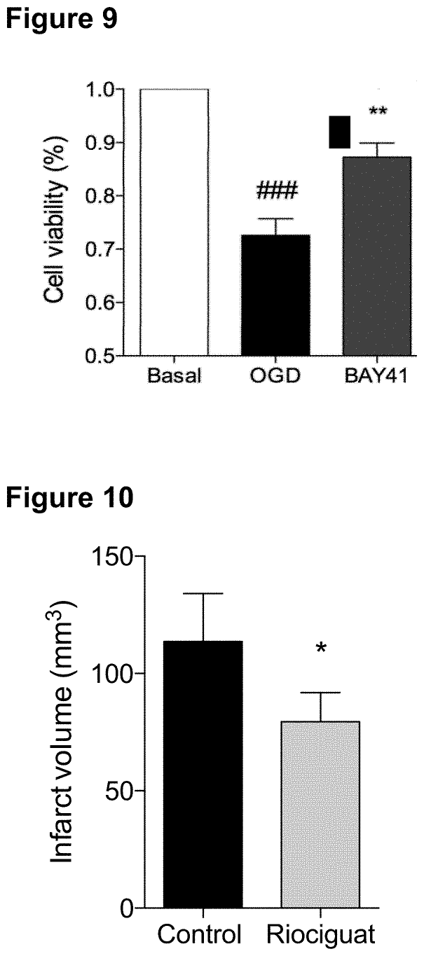

FIG. 7A shows the sGC activity of purified human sGC (EnzoLifeScience, Lörrach, Germany) treated with 10 μM ODQ and stimulated with the sGC activator BAY 58-2667 at 0.3 μM alone (open bar); and in presence of sGC stimulator BAY 41-2272 at 10 μM (black bar). The specific activity of sGC is expressed in μmol/mg/min. *P<0.05 Student's-t-test; data represents means±standard error mean from three experiments. FIG. 7B shows the dose response curve for the purified human sGC treated with a concentration series of ODQ in the presence of 30 μM of the sGC stimulator Bay 41-2272. These data altogether show that human sGC which comprises fully oxidized heme (the amount of ODQ tested is far beyond the amount required to obtain sGC with 100% oxidized heme), is still activatable by potentiation with an sGC stimulator, here Bay 41-2272. Without wishing to be bound by any theory, the sGC stimulator activates a second NO binding site that is independent of the (oxidized) heme group, and that is susceptible for potentiation by sGC stimulators although in the apo-sGC all heme is oxidized: this second NO binding site is referred to as a pseudo NO binding site. In the absence of the Bay 41-2272, no cGMP is formed at all due to the inactivated apo-sGC at the doses ODQ applied on the cells.
FIG. 8 shows the sGC activity of human apo-sGC expressed in SF-9 cells stimulated with the sGC stimulator BAY 41-2272 at 10 μM alone (open bar); and in presence of ODQ at 10 μM (black bar). The specific activity of sGC is expressed in nmol/mg/min. *P<0.05 Student's-t-test; data represents means±standard error mean from three experiments. These data clearly show that although the human apo-sGC does not comprise the functional (primary) NO binding site in the heme group due to the absence of functional heme, still under influence of the sGC stimulator Bay 41-2272, the apo-sGC is activated and cGMP is (increasingly) produced. Even when the cells are contacted with ODQ, the sGC stimulator improves the formation of cGMP. Notably, in the absence of the sGC stimulator, no cGMP is formed at all.
FIG. 9 shows the cGMP production stimulatory effect of an sGCs in the apo-sGC in vitro disease model, for stroke. Human Brain Microvascular Endothelial Cells (HBMECs) were subjected to hypoxia and treated with sGCs BAY 41-2272 (‘BAY41’, 1 μM). sGCs treatment (grey bar, right) significantly increased cell viability and restored cell viability to a level comparable to control cells that were untreated (left bar, white) and in comparison with non-treated cells, which were treated with oxygen-glucose deprivation (OGD) (black bar, middle). ^{###}<0.001 and **P<0.01 Student's-t-test; data represents means±standard error mean from six experiments. These results show that an sGCs stimulator still is efficient in stimulating cGMP production by apo-sGC that is formed as a result of the OGD, and which thus as a consequence does not contain the NO sensible heme group. Surprisingly, the apo-sGC is still activatable by sensitizing the apo-sGC for NO binding (likely, due to binding of the NO to the heme-independent secondary pseudo-NO binding site in sGC and apo-sGC).

FIG. 10 shows that the post-stroke treatment with sGC stimulator BAY 63-2521 (riociguat, approved as Adempas) reduces infarct size in a stroke animal model. Adult mice were subjected to 1 h transient occlusion of the middle cerebral artery (tMCAO) followed by 24 h of reperfusion. 1 h post-reperfusion treatment with the sGCs reduced infarct volume (grey bar, right) in comparison to non-treated animals (black bar, left), and *P<0.05 Student's-t-test; data represents means±standard error mean from six experiments. These results show that the apo-sGC formed due to the ischemic conditions and hypoxia as a result of the transient occlusion, is still susceptible to stimulation by an sGCs, although no active NO-binding heme group is present. This shows that although the sGC is apo-sGC comprising inactive heme with regard to NO binding, still cGMP synthesis can be stimulated by potentiating the apo-sGC with the sGCs.

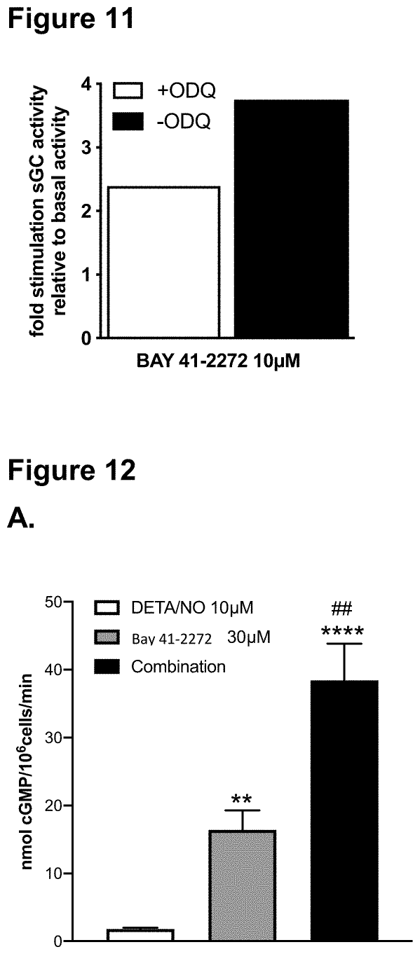

FIG. 11 shows the sGC activity in lung homogenates of apo-sGC mice stimulated with the sGC stimulator BAY 41-2272 at 10 μM in presence (open bar, left); and in absence of ODQ at 10 μM (black bar, right). Notably, in the absence of ODQ and Bay 41-2272, and in the presence of ODQ, no cGMP was formed, Only upon stimulation of the apo-sGC with the sGCs, cGMP is synthesized. This again shows that sGCs compounds formerly known as being effective when patient have sGC, are also effective under disease conditions accompanied by the occurrence and abundant presence of apo-sGC, or even under conditions wherein sGC is absent. Binding of an sGCs to apo-sGC improves the NO-binding driven formation of cGMP in a heme independent manner. That is to say, absence of heme or presence of oxidized heme such as apparent under conditions of ischemia, neonatal asphyxia, stroke, hypoxia, acute respiratory distress syndrome, asthma, oxidative (organ and/or tissue and/or cell) damage, etc., does not hamper the beneficial and stimulatory effect of an sGCs with regard to the cGMP production. Thus, even under conditions wherein an sGC activator does not exert or does not sufficiently exert the desired effect when cGMP production by apo-sGC is considered, a new treatment option has now become apparent due to the invention, i.e. treating the patient with one or more sGC stimulators such as riociguat, Bay 41-2272, vericiguat, Bay 12-11163, either or not in combination with an NO donor and/or either or not in combination with an sGC activator.

FIG. 12 shows the production of cGMP by Sf9 cells which were co-infected with baculoviruses expressing human sGCa1 and mutant sGCβ1H105F (apo-sGC). Forty-eight hours post-infection, the cells were treated with vehicle, NO donor, sGC stimulator, sGC activator or combination in 2 ml final volume of 10⁶ cells per ml and subsequently, the cells were incubated for 10 minutes at 27° C. The reaction was terminated by the addition of 0.1M of HCl, and cGMP was extracted on ice for 20 minutes. Cells were then sonicated to ensure complete lysis. The obtained cell extract was then centrifuged and used for cGMP determination by an ELISA. A. The nitric oxide donor DETA-NONOate at 10 micromole/L concentration was added to control cells 1 (left bar, white) and to test cells (right, black bar); to the control cells 2 (middle bar, gray) and to the test cells, 30 micromole/L of Bay 41-2272 was added, resulting in an increase in cGMP formation for the control cells 2; thus, to the test cells (right bar, black), combination of 30 micromole/L of sGC stimulator Bay 41-2272 and 10 micromole/L of the NO donor compound were added, resulting in a synergistic increase in cGMP formation, compared to control cells 1 (NO donor compound only) and compared to control cells 2 (Bay 41-2272, only). Surprisingly, the apo-sGC is susceptible for stimulation of cGMP synthesis by the sGCs. Contacting the cells with the NO donor only has no effect on cGMP formation. In the absence of any of the NO donor, the sGCs or the combination of these compounds, no cGMP synthesis is detected. It is now for the first time shown by the inventors that apo-sGC is still activatable even in the absence of an sGC activator known in the art. The presence of the sGCs facilitates NO binding to a secondary, heme-independent NO binding-site, and in addition, presence of an NO donor acts synergistically, when cGMP production is considered. Due to the invention, it is now made possible to treat patients with an sGCs, resulting in cGMP formation, under conditions wherein treatment with sGC activators has no effect at all or a too small effect, e.g. under conditions of ischemia, neonatal asphyxia, stroke, hypoxia, acute respiratory distress syndrome, asthma, oxidative (organ and/or tissue and/or cell) damage, etc. The invention provides a new treatment modality for these patients, which are often not sensitive (enough) for sGCa treatment. B. The nitric oxide donor DETA-NONOate at 10 micromole/L concentration was added to control cells (left bar) and to test cells (right, black bar); to the test cells, also 30 micromole/L of Bay 41-2272 was added, resulting in an increase in cGMP formation. C. The nitric oxide donor DETA-NONOate at 100 micromole/L concentration and Bay 41-2272 at 30 micromole/L concentration were added to control cells (left bar) and to test cells (right, black bar); to the test cells, also 0.1 micromole/L of Bay60-2770 was added, resulting in a similar extent of cGMP formation compared to the control cells (left bar, gray). These results, also in the context of the test results displayed in FIGS. 12A and B, show that at an optimal dose of the NO donor and the sGC stimulator, which act synergistically when cGMP synthesis is considered, no further stimulation of cGMP production is achieved when the cells comprising apo-sGC are further contacted with an sGC activator.

Briefly summarizing:

It is part of the invention that sGCs can now be administered to patients suffering from conditions relating to the presence of apo-sGC. It is also part of the invention that an sGCs and an sGCa act synergistically when administered in combination to patients suffering from conditions relating to the presence of apo-sGC. Such conditions are typically patients after stroke, patients suffering from any one or more of ischemia, neonatal asphyxia, oxidative organ damage, oxidative tissue damage, oxidative cell damage, stroke, acute respiratory distress syndrome and asthma, preferably any one or more of ischemia, neonatal asphyxia, oxidative organ damage, oxidative tissue damage, oxidative cell damage and stroke, more preferably ischemia and stroke, and optionally wherein the patient suffers from a disease or disorder accompanied by the presence of apo-sGC and the absence of sGC. Typical examples, which are preferred combinations, are a single sGCs selected from riociguat and vericiguat, or the combination thereof, either or not in combination with an sGCa such as Bay 12-11163, for the treatment of conditions relating to the presence of apo-sGC, or to the presence of sGC with oxidized heme or sGC wherein heme is absent in the patient to be treated. Optionally and preferably, the patient suffering from too low or absent cGMP synthesis, e.g. due to presence of apo-sGC and/or low or absent sGC, is treated with a combination of one or more, such as one or two sGCs, optionally one or more, such as a single or two sGCa, and at least one NO donor.

1. Pharmaceutical composition comprising a soluble guanylate cyclase (sGC) stimulator compound (sGCs) and an sGC activator compound (sGCa).

2. Pharmaceutical composition according to claim 1, comprising at least two sGCs and at least one sGCa, or one sGCs and at least two sGCa.

3. Pharmaceutical composition of claim 1 or 2, wherein the sGCs is/are any one or more of the sGCs listed in Table 3 and/or the sGCa is/are any one or more of the sGCa listed in Table 4.

4. Pharmaceutical composition of claim 1 or 2, wherein the sGCs is/are any one or more of riociguat (BAY 63-2521), vericiguat (BAY 1021189/MK-1242-001), nelociguat (desmethyl riociguat), olinciguat (IW-1701), BAY 41-2272, BAY 60-4552, IWP-953, A-350619, CF-1571, CFM-1571, lificiguat (YC-1), etriciguat, praliciguat (IW-1973), preferably one or two of riociguat (BAY 63-2521) and vericiguat (BAY 1021189/MK-1242-001), and/or the sGCa is/are any one or more of cinaciguat (BAY 58-2667), BAY 60-2770, ataciguat (HMR 1766), BI 703704, BI 684067, S-3448, BR-11257, MGV-354, TY-55002 and Bay 12-11163, preferably Bay 12-11163, preferably the sGCs is riociguat and the sGCa is Bay 12-11163 or the sGCs is vericiguat and the sGCa is Bay 12-1116.

5. Pharmaceutical composition of any one of the claims 1-4, wherein the sGCs is at least one of riociguat (BAY 63-2521), nelociguat (BAY 60-4552), vericiguat (BAY 102-1189), olinciguat (IW-1701), praliciguat (IW-1973), and wherein the sGCa is ataciguat (HMR 1766) or Bay 12-1116.

6. Pharmaceutical composition of any one of the claims 1-5, wherein the sGCs is provided as a unit dose comprising 0.05 mg-100 mg of the one or more sGCs, such as 0.1 mg-50 mg, preferably 0.2 mg-25 mg, more preferably 0.4 mg-10 mg, most preferably 1 mg-5 mg, such as 2 mg-3 mg, and/or wherein the sGCa is provided as a unit dose comprising 0.5 mg-500 mg of the one or more sGCa or ataciguat when depending on claim 5, preferably 1 mg-300 mg, more preferably 2 mg-200 mg, most preferably 3 mg-100 mg, such as 4 mg-50 mg, or 5 mg-25 mg.

7. Pharmaceutical composition of any one of the claims 1-6, wherein the pharmaceutical composition further comprises a further active pharmaceutical ingredient.

8. Pharmaceutical composition of any one of the claims 1-7, wherein the sGCs is provided as a unit dose comprising 0.1 mg-5 mg riociguat, preferably less than 2.5 mg, 0.05 mg-2 mg nelociguat, preferably less than 1 mg, 0.1 mg-30 mg vericiguat, preferably less than 15 mg, 0.1 mg-10 mg olinciguat, preferably less than 5 mg, 1 mg-100 mg praliciguat, preferably less than 50 mg, and/or wherein the sGCa is provided as a unit dose comprising 5 mg-400 mg ataciguat, preferably less than 200 mg.

9. Pharmaceutical composition according to any one of the claims 1-8, further comprising at least one nitric-oxide (NO) donor compound.

10. Pharmaceutical composition according to claim 9, wherein the at least one NO donor compound is any one or more compound(s) selected from an organic nitrate, an organic nitrite, an S-nitrosothiol, a prusside, an NONOate, a sydnonimine, an oxatriazole, a furoxan, a Ruthenium nitrosyl, a photochemical donor via one/two-photon excitation, a diazeniumdiolated carbamate, nitroglycerin, molsidomine, isosorbide dinitrate (ISDN), sodium nitroprusside or an alternative pharmaceutically acceptable nitroprusside salt, and any pharmaceutically acceptable derivative thereof and/or any pharmaceutically acceptable salt thereof and/or any pharmaceutically acceptable prodrug thereof.

11. Oral dose combination comprising a first oral dose comprising an sGCs and a second oral dose comprising an sGCa, the first oral dose and the second oral dose optionally comprising one or more pharmaceutically acceptable excipient(s).

12. Oral dose combination of claim 11, wherein the sGCs is an sGCs according to any one of the claims 1-5 and/or wherein the sGCs is an sGCs of claim 6 or 7 provided at the dose of claim 6 or 7, and/or wherein the sGCa is an sGCa according to any one of the claims 1-5 and/or wherein the sGCa is an sGCa of claim 6 or 7 provided at the dose of claim 6 or 7.

13. Oral dose combination of claim 11 or 12, further comprising a third oral dose comprising an NO donor compound, preferably an NO donor compound according to claim 10.

14. Pharmaceutical composition of any one of the claims 1-10 wherein the sGCs and the sGCa are provided as a solid dosage form such as a capsule or a tablet, and when dependent on claim 9 or 10, wherein the NO donor compound is provided as a solid dosage form, or oral dose combination of any one of the claims 11-13, wherein the sGCs and the sGCa each are provided separately as a solid dosage form such as a capsule or a tablet, and when dependent on claim 13, wherein the NO donor compound is provided separately from the sGCs and the sGCa as a solid dosage form.

15. Pharmaceutical composition of claim 14 wherein a single unit of the solid dosage form contains a daily dosage of the one or more sGCs and the one or more sGCa and if present the one or more NO donor compound(s), or oral dose combination of claim 14, wherein a single unit of the solid dosage form containing the sGCs contains a daily dosage of the one or more sGCs and/or a single unit of the solid dosage form containing the sGCa contains a daily dosage of the one or more sGCa and if present the one or more NO donor compound(s).

16. Kit comprising the pharmaceutical composition of any one of the claim 1-10, 14 or 15 or the oral dose combination of any one of the claims 11-15, and optionally instructions for use.

17. Pharmaceutical composition of any one of the claim 1-10 or 14-16 or oral dose combination of any one of the claims 11-16, for use as a medicament.

18. Pharmaceutical composition or oral dose combination for use according to claim 17, wherein the use is in a method for the treatment of cyclic 3′,5′-guanosine monophosphate (cGMP) deficiency in a patient, preferably a human patient.

19. Pharmaceutical composition or oral dose combination for use according to claim 17 or 18, wherein the use is in a method for the treatment of a cardiovascular disease, or wherein the patient deficient in cGMP suffers from a cardiovascular disease.

20. Pharmaceutical composition or oral dose combination for use according to any one of the claims 17-19, wherein the patient to whom the pharmaceutical composition or the oral dose combination is administered, suffers from any one or more of pulmonary arterial hypertension, chronic thromboembolic pulmonary hypertension, pulmonary hypertension, persistent pulmonary hypertension of the new born, portal hypertension, pulmonary hypertension—left ventricular systolic dysfunction, pulmonary hypertension—idiopathic interstitial pneumonias, diffuse cutaneous systemic sclerosis, cystic fibrosis, moyamoya syndrome, sickle cell disease, erectile dysfunction, heart failure with reduced ejection fraction, heart failure with preserved ejection fraction, type 2 diabetes mellitus, hypertension, acute decompensated chronic congestive heart failure, moderate calcific aortic valve stenosis, peripheral arterial disease, erectile dysfunction, fibrotic conditions such as liver fibrosis, NASH, complications relating to diabetes mellitus, such as diabetic nephropathy and diabetic cardiomyopathy, and COVID19-related respiratory distress and/or cardiovascular complications, ischemia, neonatal asphyxia, oxidative organ damage, oxidative tissue damage, oxidative cell damage, stroke, acute respiratory distress syndrome and asthma, preferably ischemia, neonatal asphyxia, oxidative organ damage, oxidative tissue damage, oxidative cell damage and stroke, more preferably ischemia and stroke.

21. Pharmaceutical composition or oral dose combination for use according to any one of the claims 17-20, wherein the pharmaceutical composition is administered to the patient as a single unit dose daily, or as two-four unit doses daily, such as thrice daily, or wherein the first oral dose and/or the second oral dose of the oral dose combination is administered to the patient as a single solid dosage daily, or as two-four solid dosages daily, such as thrice daily.

22. Pharmaceutical composition or oral dose combination for use according to any one of the claims 17-21, wherein the patient in need thereof is administered an effective dose of the pharmaceutical composition of any one of the claim 1-10 or 14-16 or is administered an effective dose of the oral dose combination of any one of the claims 11-16.

23. Pharmaceutical composition or oral dose combination for use according to any one of the claims 17-22, wherein the patient suffers from a disease or disorder accompanied by the presence of apo-sGC, such as any one or more of ischemia, neonatal asphyxia, oxidative organ damage, oxidative tissue damage, oxidative cell damage, stroke, acute respiratory distress syndrome and asthma, preferably ischemia, neonatal asphyxia, oxidative organ damage, oxidative tissue damage, oxidative cell damage and stroke, more preferably ischemia and stroke, and optionally wherein the patient suffers from a disease or disorder accompanied by the presence of apo-sGC and the absence of sGC.

24. Pharmaceutical composition or oral dose combination for use according to any one of the claims 17-23, wherein the treatment comprises stimulation of cGMP formation in the patient in need of said treatment.

25. Pharmaceutical composition or oral dose combination for use according to any one of the claims 17-24, wherein the patient suffers from nitric oxide (NO) insufficiency and/or from any one or more of oxidative damage, ischemia, neonatal asphyxia, oxidative organ damage, oxidative tissue damage, oxidative cell damage, stroke, acute respiratory distress syndrome and asthma, preferably from any one or more of nitric oxide (NO) insufficiency and/or ischemia, neonatal asphyxia, oxidative organ damage, oxidative tissue damage, oxidative cell damage and stroke, more preferably from nitric oxide (NO) insufficiency and/or ischemia and/or stroke.

26. Pharmaceutical composition or oral dose combination for use according to any one of the claims 17-25, wherein the patient suffers from a medical condition relating to sGC dysfunction and/or relating to cGMP deficiency.

27. Pharmaceutical composition or oral dose combination for use according to any one of the claims 17-26, wherein the sGCs augment(s) stimulation of heme containing sGC and augment(s) stimulation of sGC by NO and/or stimulate(s) apo-sGC.

28. Pharmaceutical composition or oral dose combination for use according to any one of the claims 17-27, wherein the one or more sGCs and the one or more sGCa augment sGC and/or apo-sGC synergistically.

29. Pharmaceutical composition comprising an sGCs for use as a medicament.

30. Pharmaceutical composition comprising at least two sGCs for use as a medicament.

31. Pharmaceutical composition for use of claim 29 or 30, wherein the sGCs is an sGCs listed in Table 3 or wherein the sGCs are any two or more of the sGCs listed in Table 3.

32. Pharmaceutical composition for use of any one of claims 29-31, wherein the sGCs is/are any one or any two or more of riociguat (BAY 63-2521), vericiguat (BAY 1021189/MK-1242-001), nelociguat (desmethyl riociguat), olinciguat (IW-1701), BAY 41-2272, BAY 60-4552, BAY 63-2521, IWP-953, A-350619, CF-1571, CFM-1571, lificiguat (YC-1), etriciguat, praliciguat (IW-1973), preferably one or two of riociguat (BAY 63-2521) and vericiguat (BAY 1021189/MK-1242-001).

33. Pharmaceutical composition for use of any one of the claims 29-32, wherein the sGCs is/are one or at least two of riociguat (BAY 63-2521), nelociguat (BAY 60-4552), vericiguat (BAY 102-1189), olinciguat (IW-1701), praliciguat (IW-1973), BAY 41-2272.

34. Pharmaceutical composition for use of any one of the claims 29-33, wherein the sGCs is/are provided as a unit dose comprising 0.05 mg-100 mg of the one or two or more sGCs, such as 0.1 mg-50 mg, preferably 0.2 mg-25 mg, more preferably 0.4 mg-10 mg, most preferably 1 mg-5 mg, such as 2 mg-3 mg.

35. Pharmaceutical composition for use of any one of the claims 29-34, wherein the pharmaceutical composition further comprises a further active pharmaceutical ingredient.

36. Pharmaceutical composition for use of any one of the claims 29-35, wherein the sGCs is/are provided as a unit dose comprising any one or any two or more of 0.1 mg-5 mg riociguat, preferably less than 2.5 mg, 0.05 mg-2 mg nelociguat, preferably less than 1 mg, 0.1 mg-30 mg vericiguat, preferably less than 15 mg, 0.1 mg-10 mg olinciguat, preferably less than 5 mg, 1 mg-100 mg praliciguat, preferably less than 50 mg.

37. Pharmaceutical composition for use of any one of the claims 29-36, wherein the pharmaceutical composition further comprises at least one nitric-oxide (NO) donor compound.

38. Pharmaceutical composition for use according to claim 37, wherein the at least one NO donor compound is any one or more compound(s) selected from an organic nitrate, an organic nitrite, an S-nitrosothiol, a prusside, an NONOate, a sydnonimine, an oxatriazole, a furoxan, a Ruthenium nitrosyl, a photochemical donor via one/two-photon excitation, a diazeniumdiolated carbamate, nitroglycerin, molsidomine, isosorbide dinitrate (ISDN), sodium nitroprusside or an alternative pharmaceutically acceptable nitroprusside salt, and any pharmaceutically acceptable derivative thereof and/or any pharmaceutically acceptable salt thereof and/or any pharmaceutically acceptable prodrug thereof.

39. Oral dose combination comprising a first oral dose comprising a first sGCs and a second oral dose comprising a second sGCs, the first oral dose and the second oral dose optionally comprising one or more pharmaceutically acceptable excipient(s), for use according to any one of the claims 30-38.

40. Oral dose combination for use of claim 39, wherein the first sGCs is an sGCs according to any one of the claims 1-5 and/or wherein the first sGCs is an sGCs of claim 6 or 7 provided at the dose of claim 6 or 7, and/or wherein the second sGCs is a second sGCs according to any one of the claims 1-5 and different from the first sGCs and/or wherein the second sGCs is an sGCs of claim 6 or 7 different from the first sGCs provided at the dose of claim 6 or 7.

41. Oral dose combination of claim 39 or 40, further comprising a third oral dose comprising an NO donor compound, preferably an NO donor compound according to claim 38.

42. Oral dose combination comprising a first oral dose comprising an sGCs and a second oral dose comprising an NO donor compound, preferably an NO donor compound according to claim 38, the first oral dose and the second oral dose optionally comprising one or more pharmaceutically acceptable excipient(s), for use according to any one of the claims 30-38.

43. Pharmaceutical composition for use of any one of the claims 29-38 wherein the sGCs is/are provided as a solid dosage form such as a capsule or a tablet, and when dependent on claim 37 or 38, wherein the at least one NO donor compound is provided separately as a solid dosage form, or oral dose combination for use of any one of the claims 39-41, wherein the first sGCs and the second sGCs each are provided separately as a solid dosage form such as a capsule or a tablet, and when dependent on claim 41 or 42, wherein the at least one NO donor compound is provided separately as a solid dosage form.

44. Pharmaceutical composition for use of claim 43 wherein a single unit of the solid dosage form contains a daily dosage of the one or the two or more sGCs, or oral dose combination for use of claim 43, wherein a single unit of the solid dosage form containing the first sGCs contains a daily dosage of the first sGCs and/or a single unit of the solid dosage form containing the second sGCs contains a daily dosage of the second sGCs, and if present, wherein a single unit of the solid dosage form containing the NO donor compound contains a daily dosage of the NO donor compound.

45. Pharmaceutical composition or oral dose combination for use according to any one of the claims 29-44, wherein the use is in a method for the treatment of cyclic 3′,5′-guanosine monophosphate (cGMP) deficiency in a patient, preferably a human patient.

46. Pharmaceutical composition or oral dose combination for use according to any one of the claims 29-45, wherein the use is in a method for the treatment of a cardiovascular disease, or wherein the patient deficient in cGMP suffers from a cardiovascular disease.

47. Pharmaceutical composition or oral dose combination for use according to any one of the claims 29-46, wherein the patient to whom the pharmaceutical composition or the oral dose combination is administered, suffers from any one or more of pulmonary arterial hypertension, chronic thromboembolic pulmonary hypertension, pulmonary hypertension, persistent pulmonary hypertension of the new born, portal hypertension, pulmonary hypertension—left ventricular systolic dysfunction, pulmonary hypertension—idiopathic interstitial pneumonias, diffuse cutaneous systemic sclerosis, cystic fibrosis, moyamoya syndrome, sickle cell disease, erectile dysfunction, heart failure with reduced ejection fraction, heart failure with preserved ejection fraction, type 2 diabetes mellitus, hypertension, acute decompensated chronic congestive heart failure, moderate calcific aortic valve stenosis, peripheral arterial disease, erectile dysfunction, fibrotic conditions such as liver fibrosis, NASH, complications relating to diabetes mellitus, such as diabetic nephropathy and diabetic cardiomyopathy, and COVID19-related respiratory distress and/or cardiovascular complications, and/or suffers from any one or more of oxidative damage, ischemia, neonatal asphyxia, oxidative organ damage, oxidative tissue damage, oxidative cell damage, stroke, acute respiratory distress syndrome and asthma, preferably from any one or more of nitric oxide (NO) insufficiency and/or ischemia, neonatal asphyxia, oxidative organ damage, oxidative tissue damage, oxidative cell damage and stroke, more preferably from nitric oxide (NO) insufficiency and/or ischemia and/or stroke.

48. Pharmaceutical composition or oral dose combination for use according to any one of the claims 29-47, wherein the pharmaceutical composition is administered to the patient as a single unit dose daily, or as two-four unit doses daily, such as thrice daily, or wherein the first oral dose and/or the second oral dose of the oral dose combination is administered to the patient as a single solid dosage daily, or as two-four solid dosages daily, such as thrice daily.

49. Pharmaceutical composition or oral dose combination for use according to any one of the claims 29-48, wherein the patient in need thereof is administered an effective dose of the pharmaceutical composition of any one of the claims 29-38, 43-48 or is administered an effective dose of the oral dose combination of any one of the claims 39-48.

50. Pharmaceutical composition or oral dose combination for use according to any one of the claims 29-49, wherein the patient suffers from a disease or disorder accompanied by the presence of apo-sGC, such as any one or more of ischemia, neonatal asphyxia, oxidative organ damage, oxidative tissue damage, oxidative cell damage, stroke, acute respiratory distress syndrome and asthma, preferably ischemia, neonatal asphyxia, oxidative organ damage, oxidative tissue damage, oxidative cell damage and stroke, more preferably ischemia and stroke, and optionally wherein the patient suffers from a disease or disorder accompanied by the presence of apo-sGC and the absence of sGC.

51. Pharmaceutical composition or oral dose combination for use according to any one of the claims 29-50, wherein the treatment comprises stimulation of cGMP formation in the patient in need of said treatment.

52. Pharmaceutical composition or oral dose combination for use according to any one of the claims 29-51, wherein the patient suffers from any one or more of nitric oxide (NO) insufficiency and/or from oxidative damage, ischemia, neonatal asphyxia, oxidative organ damage, oxidative tissue damage, oxidative cell damage, stroke, acute respiratory distress syndrome and asthma, preferably from any one or more of nitric oxide (NO) insufficiency and/or ischemia, neonatal asphyxia, oxidative organ damage, oxidative tissue damage, oxidative cell damage and stroke, more preferably from nitric oxide (NO) insufficiency and/or ischemia and/or stroke.

53. Pharmaceutical composition or oral dose combination for use according to any one of the claims 29-52, wherein the patient suffers from a medical condition relating to sGC dysfunction and/or relating to cGMP deficiency.

54. Pharmaceutical composition or oral dose combination for use according to any one of the claims 29-53, wherein the sGCs augment(s) stimulation of heme containing sGC and augment(s) stimulation of sGC by NO and/or stimulate(s) apo-sGC.

55. Pharmaceutical composition or oral dose combination for use according to any one of the claims 30-54, wherein the two or more sGCs augment sGC and/or apo-sGC synergistically.

56. Therapeutic combination comprising:
a. a first unit dose comprising:
i. a first sGCs;
ii. optionally a second sGCs; and either
b. a second unit dose comprising:
i. a first sGCa;
ii. optionally a second sGCa; or
c. a third unit dose comprising:
i. a third sGCs; and
ii. optionally a fourth sGCs.

57. Therapeutic combination of claim 56, wherein the first, second, third and fourth sGCs is any of Riociguat, Vericiguat, BAY 60-4552, YC-1, A-350619, CF-1571, Olinciguat, Praliciguat, wherein the first and second sGCa is any of Cinaciguat, HMR 1766, BI 703704, BI 684067 and Bay 12-11163, preferably the sGCs is Riociguat or Vericiguat and the sGCa is Bay 12-11163.

58. Therapeutic combination of claim 56 or 57, wherein the amount sGCs per unit dose in the first and/or third unit dose is for riociguat 5 mg or lower, preferably less than 4 mg, less than 3 mg, less than 2.5 mg, less than 2.0 mg, less than 1.5 mg, less than 1.0 mg, preferably less than 0.5 mg; for nelociguat 1.0 mg or less such as less than 0.8 mg, less than 0.5 mg; for vericiguat 15 mg or less such as less than 10 mg, less than 5 mg, less than 2.5 mg, less than 1.25 mg such as 0.2 mg-1.0 mg; for olinciguat 5 mg or less such as less than 4 mg, less than 3 mg, such as 0.5 mg-2.5 mg; of praliciguat 50 mg or less such as less than 40 mg, less than 30 mg, less than 20 mg, less than 10 mg, such as 1 mg-6 mg; and wherein the amount sGCa per unit dose in the second unit dose is for ataciguat 200 mg/dose or less, preferably less than 100 mg/dose, less than 50 mg/dose, less than 40 mg/dose, less than 30 mg/dose, less than 20 mg/dose, less than 15 mg/dose, less than 10 mg/dose, preferably less than 5 mg/dose.

59. Therapeutic combination of any one of the claims 56-58, comprising a first unit dose and a second unit dose, the first and second unit dose comprising respectively any combination of Riociguat, Cinaciguat; Riociguat, HMR 1766; Riociguat, BI 703704; Riociguat, BI 684067; Riociguat, Bay 12-11163; Vericiguat, Cinaciguat; Vericiguat, HMR 1766; Vericiguat, BI 703704; Vericiguat, BI 684067; Vericiguat, Bay 12-11163; BAY 60-4552, Cinaciguat; BAY 60-4552, HMR 1766; BAY 60-4552, BI 703704; BAY 60-4552, BI 684067; BAY 60-4552, Bay 12-11163; YC-1, Cinaciguat; YC-1, HMR 1766; YC-1, BI 703704; YC-1, BI 684067; YC-1, Bay 12-11163; A-350619, Cinaciguat; A-350619, HMR 1766; A-350619, BI 703704; A-350619, BI 684067; CF-1571, Cinaciguat; CF-1571, HMR 1766; CF-1571, BI 703704; CF-1571, BI 684067; Olinciguat, Cinaciguat; Olinciguat, HMR 1766; Olinciguat, BI 703704; Olinciguat, BI 684067; Praliciguat, Cinaciguat; Praliciguat, HMR 1766; Praliciguat, BI 703704; or Praliciguat, BI 684067; or comprising a first unit dose and a third unit dose, the first and third unit dose comprising respectively any combination of Riociguat, Vericiguat; Riociguat, BAY 60-4552; Riociguat, YC-1; Riociguat, A-350619; Riociguat, CF-1571; Riociguat, Olinciguat; Riociguat, Praliciguat; Vericiguat, BAY 60-4552; Vericiguat, YC-1; Vericiguat, A-350619; Vericiguat, CF-1571; Vericiguat, Olinciguat; Vericiguat, Praliciguat; BAY 60-4552, YC-1; BAY 60-4552, A-350619; BAY 60-4552, CF-1571; BAY 60-4552, Olinciguat; BAY 60-4552, Praliciguat; YC-1, A-350619; YC-1, CF-1571; YC-1, Olinciguat; YC-1, Praliciguat; A-350619, CF-1571; A-350619, Olinciguat; A-350619, Praliciguat; CF-1571, Olinciguat; CF-1571, Praliciguat; or Olinciguat, Praliciguat.

60. Therapeutic combination of any one of the claims 56-59, for use as a medicament.

61. Therapeutic combination of any one of the claims 56-59 for use of claim 60, wherein the use is in a method for the treatment of cyclic 3′,5′-guanosine monophosphate (cGMP) deficiency in a patient, preferably a human patient.

62. Therapeutic combination for use according to claim 60 or 61, wherein the use is in a method for the treatment of a cardiovascular disease, or wherein the patient deficient in cGMP suffers from a cardiovascular disease.

63. Therapeutic combination for use according to any one of the claims 60-62, wherein the patient to whom the therapeutic combination is administered, suffers from any one or more of pulmonary arterial hypertension, chronic thromboembolic pulmonary hypertension, pulmonary hypertension, persistent pulmonary hypertension of the new born, portal hypertension, pulmonary hypertension—left ventricular systolic dysfunction, pulmonary hypertension—idiopathic interstitial pneumonias, diffuse cutaneous systemic sclerosis, cystic fibrosis, moyamoya syndrome, sickle cell disease, erectile dysfunction, heart failure with reduced ejection fraction, heart failure with preserved ejection fraction, type 2 diabetes mellitus, hypertension, acute decompensated chronic congestive heart failure, moderate calcific aortic valve stenosis, peripheral arterial disease, erectile dysfunction, fibrotic conditions such as liver fibrosis, NASH, complications relating to diabetes mellitus, such as diabetic nephropathy and diabetic cardiomyopathy, and COVID19-related respiratory distress and/or cardiovascular complications, and/or suffers from any one or more of oxidative damage, ischemia, neonatal asphyxia, oxidative organ damage, oxidative tissue damage, oxidative cell damage, stroke, acute respiratory distress syndrome and asthma, preferably from any one or more of nitric oxide (NO) insufficiency and/or ischemia, neonatal asphyxia, oxidative organ damage, oxidative tissue damage, oxidative cell damage and stroke, more preferably from nitric oxide (NO) insufficiency and/or ischemia and/or stroke.

64. Therapeutic combination for use according to any one of the claims 60-63, wherein the therapeutic combination wherein the first, second, third unit dose is administered to the patient as a single solid dosage daily, or as two-four solid dosages daily, such as thrice daily, and wherein the therapeutic combination is for oral administration.

65. Therapeutic combination for use according to any one of the claims 60-64, wherein the patient in need thereof is administered an effective dose of the therapeutic combination of any one of the claims 56-59.

66. Therapeutic combination for use according to any one of the claims 60-65, wherein the patient suffers from a disease or disorder accompanied by the presence of apo-sGC, such as any one or more of ischemia, neonatal asphyxia, oxidative organ damage, oxidative tissue damage, oxidative cell damage, stroke, acute respiratory distress syndrome and asthma, preferably ischemia, neonatal asphyxia, oxidative organ damage, oxidative tissue damage, oxidative cell damage and stroke, more preferably ischemia and stroke, and optionally wherein the patient suffers from a disease or disorder accompanied by the presence of apo-sGC and the absence of sGC.

67. Therapeutic combination for use according to any one of the claims 60-66, wherein the treatment comprises stimulation of cGMP formation in the patient in need of said treatment.

68. Therapeutic combination for use according to any one of the claims 60-67, wherein the patient suffers from NO insufficiency and/or from oxidative damage, ischemia, neonatal asphyxia, oxidative organ damage, oxidative tissue damage, oxidative cell damage, stroke, acute respiratory distress syndrome and asthma, preferably from any one or more of nitric oxide (NO) insufficiency and/or ischemia, neonatal asphyxia, oxidative organ damage, oxidative tissue damage, oxidative cell damage and stroke, more preferably from nitric oxide (NO) insufficiency and/or ischemia and/or stroke.

69. Therapeutic combination for use according to any one of the claims 60-68, wherein the patient suffers from a medical condition relating to sGC dysfunction and/or relating to cGMP deficiency.

70. Therapeutic combination for use according to any one of the claims 60-69, wherein the sGCs augment(s) stimulation of heme containing sGC and augment(s) stimulation of sGC by NO and/or stimulate(s) apo-sGC.

71. Therapeutic combination for use according to any one of the claims 60-70, wherein the one or more sGCs and the one or more sGCa augment sGC and/or apo-sGC synergistically.

72. Therapeutic combination comprising:
a. a first unit dose comprising:
i. a first sGCs;
ii. optionally a second sGCs; and
b. a second unit dose comprising:
i. a first NO donor compound;
ii. optionally a second NO donor compound; and optionally comprising
c. a third unit dose comprising:
i. a first sGCa; and
ii. optionally a second sGCa.

73. Therapeutic combination of claim 72, wherein the first and if present the second sGCs is any of Riociguat, Vericiguat, BAY 60-4552, YC-1, A-350619, CF-1571, Olinciguat, Praliciguat, preferably Riociguat and/or Vericiguat, and when present, wherein the first and second sGCa is any of Cinaciguat, HMR 1766, BI 703704, BI 684067 and Bay 12-11163.

74. Therapeutic combination of claim 72 or 73, wherein the amount sGCs per unit dose in the first unit dose is for riociguat 5 mg or lower, preferably less than 4 mg, less than 3 mg, less than 2.5 mg, less than 2.0 mg, less than 1.5 mg, less than 1.0 mg, preferably less than 0.5 mg; for nelociguat 1.0 mg or less such as less than 0.8 mg, less than 0.5 mg; for vericiguat 15 mg or less such as less than 10 mg, less than 5 mg, less than 2.5 mg, less than 1.25 mg such as 0.2 mg-1.0 mg; for olinciguat 5 mg or less such as less than 4 mg, less than 3 mg, such as 0.5 mg-2.5 mg; of praliciguat 50 mg or less such as less than 40 mg, less than 30 mg, less than 20 mg, less than 10 mg, such as 1 mg-6 mg; and if present wherein the amount sGCa per unit dose in the third unit dose is for ataciguat 200 mg/dose or less, preferably less than 100 mg/dose, less than 50 mg/dose, less than 40 mg/dose, less than 30 mg/dose, less than 20 mg/dose, less than 15 mg/dose, less than 10 mg/dose, preferably less than 5 mg/dose.

75. Therapeutic combination of any one of the claims 72-74, wherein the first NO donor compound and when present the second NO donor compound is/are selected from an organic nitrate, an organic nitrite, an S-nitrosothiol, a prusside, an NONOate, a sydnonimine, an oxatriazole, a furoxan, a Ruthenium nitrosyl, a photochemical donor via one/two-photon excitation, a diazeniumdiolated carbamate, nitroglycerin, molsidomine, isosorbide dinitrate (ISDN), sodium nitroprusside or an alternative pharmaceutically acceptable nitroprusside salt, and any pharmaceutically acceptable derivative thereof and/or any pharmaceutically acceptable salt thereof and/or any pharmaceutically acceptable prodrug thereof.

76. Therapeutic combination of any one of the claims 72-75, for use as a medicament.

77. Therapeutic combination of any one of the claims 72-75 for use of claim 76, wherein the use is in a method for the treatment of cyclic 3′,5′-guanosine monophosphate (cGMP) deficiency in a patient, preferably a human patient.

78. Therapeutic combination for use according to claim 76 or 77, wherein the use is in a method for the treatment of a cardiovascular disease, or wherein the patient deficient in cGMP suffers from a cardiovascular disease.

79. Therapeutic combination for use according to any one of the claims 76-78, wherein the patient to whom the therapeutic combination is administered, suffers from any one or more of pulmonary arterial hypertension, chronic thromboembolic pulmonary hypertension, pulmonary hypertension, persistent pulmonary hypertension of the new born, portal hypertension, pulmonary hypertension—left ventricular systolic dysfunction, pulmonary hypertension—idiopathic interstitial pneumonias, diffuse cutaneous systemic sclerosis, cystic fibrosis, moyamoya syndrome, sickle cell disease, erectile dysfunction, heart failure with reduced ejection fraction, heart failure with preserved ejection fraction, type 2 diabetes mellitus, hypertension, acute decompensated chronic congestive heart failure, moderate calcific aortic valve stenosis, peripheral arterial disease, erectile dysfunction, fibrotic conditions such as liver fibrosis, NASH, complications relating to diabetes mellitus, such as diabetic nephropathy and diabetic cardiomyopathy, and COVID19-related respiratory distress and/or cardiovascular complications, and/or suffers from any one or more of oxidative damage, ischemia, neonatal asphyxia, oxidative organ damage, oxidative tissue damage, oxidative cell damage, stroke, acute respiratory distress syndrome and asthma, preferably from any one or more of nitric oxide (NO) insufficiency and/or ischemia, neonatal asphyxia, oxidative organ damage, oxidative tissue damage, oxidative cell damage and stroke, more preferably from nitric oxide (NO) insufficiency and/or ischemia and/or stroke.

80. Therapeutic combination for use according to any one of the claims 76-79, wherein the therapeutic combination wherein the first, second, third unit dose is administered to the patient as a single solid dosage daily, or as two-four solid dosages daily, such as thrice daily, and wherein the therapeutic combination is for oral administration.

81. Therapeutic combination for use according to any one of the claims 76-80, wherein the patient in need thereof is administered an effective dose of the therapeutic combination of any one of the claims 72-75.

82. Therapeutic combination for use according to any one of the claims 76-81, wherein the patient suffers from a disease or disorder accompanied by the presence of apo-sGC, such as any one or more of ischemia, neonatal asphyxia, oxidative organ damage, oxidative tissue damage, oxidative cell damage, stroke, acute respiratory distress syndrome and asthma, preferably ischemia, neonatal asphyxia, oxidative organ damage, oxidative tissue damage, oxidative cell damage and stroke, more preferably ischemia and stroke, and optionally wherein the patient suffers from a disease or disorder accompanied by the presence of apo-sGC and the absence of sGC.

83. Therapeutic combination for use according to any one of the claims 76-82, wherein the treatment comprises stimulation of cGMP formation in the patient in need of said treatment.

84. Therapeutic combination for use according to any one of the claims 76-83, wherein the patient suffers from NO insufficiency and/or from oxidative damage, ischemia, neonatal asphyxia, oxidative organ damage, oxidative tissue damage, oxidative cell damage, stroke, acute respiratory distress syndrome and asthma, preferably from any one or more of nitric oxide (NO) insufficiency and/or ischemia, neonatal asphyxia, oxidative organ damage, oxidative tissue damage, oxidative cell damage and stroke, more preferably from nitric oxide (NO) insufficiency and/or ischemia and/or stroke.

85. Therapeutic combination for use according to any one of the claims 76-84, wherein the patient suffers from a medical condition relating to sGC dysfunction and/or relating to cGMP deficiency.

86. Therapeutic combination for use according to any one of the claims 76-85, wherein the sGCs augment(s) stimulation of heme containing sGC and augment(s) stimulation of sGC by NO and/or stimulate(s) apo-sGC.

87. Therapeutic combination for use according to any one of the claims 76-86, wherein the one or more sGCs and the one or more sGCa augment sGC and/or apo-sGC synergistically.
